(19) United States
(12) Patent Application Publication
(10) Pub. No.: US 2025/0283124 A1
(43) Pub. Date: Sep. 11, 2025

(54) POLYHYDROXYALKANOATE-PRODUCING BACTERIA AND METHODS FOR MAKING AND USING THEM

(71) Applicants: SAN DIEGO STATE UNIVERSITY (SDSU) FOUNDATION, DBA SAN DIEGO STATE UNIVERSITY RESEARCH FOUNDATION, SAN DIEGO, CA (US); PHAXTEC, INC., WAKE FOREST, NC (US)

(72) Inventors: Marina G. KALYUZHNAYA, SAN DIEGO, CA (US); Anindya MUKHERJEE, WAKE FOREST, NC (US)

(21) Appl. No.: 18/552,032

(22) PCT Filed: Mar. 24, 2022
(86) PCT No.: PCT/US2022/021773

§ 371 Date: Sep. 22, 2023

Related U.S. Application Data
(60) Provisional application No. 63/166,150, filed on Mar. 25, 2021.

(51) Int. Cl. C12P 7/625; C12P 39/00

(52) U.S. Cl. CPC C12P 7/625; C12P 39/00

(57) ABSTRACT

In alternative embodiments, provided are methods for selecting, isolating and recombinantly engineering methane- and hydrogen-oxidizing autotrophs, including methanotrophic bacteria, for the production of biopolymer, renewable polymer or biodegradable polymer such as polyhydroxyalkanoate (PHA) such as polyhydroxybutyrate (PHB) and co-polymers, and products of manufacture and kits, and methods for using them to produce biopolymer, renewable polymer or biodegradable polymer. Provided and isolation are efficient methane-consuming methane- and hydrogen-oxidizing autotrophic microbes for PHA (for example, PHB and co-polymers) production and methods for using them, which in alternative embodiments the methanotrophs are genetically modified to improve C1 (methane or methanol)-to PHA conversion parameters.

### RELATED APPLICATIONS

This application is a National Stage application of International Application No. PCT/US2022/021773 filed Mar. 24, 2022, which claims the benefit of priority under 35 U.S.C. § 119(e) of U.S. Provisional Application Serial No. (USSN) 63/166,150, filed Mar. 25, 2021. The aforementioned applications are expressly incorporated herein by reference in their entirety and for all purposes. All publications, patents, patent applications cited herein are hereby expressly incorporated by reference for all purposes

### TECHNICAL FIELD

This invention generally relates to bacteriology and the production of biopolymers, renewable polymers and biodegradable polymers. In alternative embodiments, provided are methods for selecting, isolating and recombinantly engineering methane- and hydrogen-oxidizing autotrophs, including methanotrophic bacteria, for the production of biopolymers, renewable polymers and biodegradable polymers such as polyhydroxyalkanoate (PHA) such as polyhydroxybutyrate (PHB) and co-polymers, and products of manufacture and kits, and methods for using them to produce biopolymers, renewable polymers and biodegradable polymers. Provided are efficient methane-consuming methane- and hydrogen-oxidizing autotrophic microbes for PHA (for example, PHB) production and methods for using them, which in alternative embodiments the methanotrophs are genetically modified to improve C1 (methane or methanol)-to PHA conversion parameters.

### BACKGROUND

Polyhydroxyalkanoates (PHA) are carbon-storage polymers produced by a variety of natural microorganisms therefore biodegradable in natural environments. PHA are a family of 100's of different polymers with beneficial attributes of the top seven best-selling plastics corresponding to a demand of over 210 million tons (or 70%) of plastics annually. PHAs are considered to be non-toxic and safe. The complete breakdown of PHA materials can be achieved in conventional industrial composters or anaerobic digesters. Overall, PHAs are envisioned as the most sustainable solution for future polymer manufacturing.

Previous attempts to produce PHAs from carbohydrates have been challenged by the high carbon feedstock cost and the low cost of competing petroleum-based and non-biodegradable polymers. Natural gas and biogas have been discussed as cost-effective alternatives, however, all established microbial platforms that produce PHAs only utilize 40% of methane carbon, while releasing the rest as CO₂. Breakthroughs in the PHA market require novel solutions for high-efficiency carbon conversion. That often means trade-offs between conventional fermentation, microbial platforms and feedstocks.

### SUMMARY

In alternative embodiments, provided are non-natural mixtures or consortium of bacteria comprising at least one member of each of the following groups:
(a) a polyhydroxyalkanoate (PHA)-producing methanotrophic bacterium or biopolymer-, renewable polymer- or biodegradable polymer-producing methanotrophic bacterium; and;
(b) a polyhydroxyalkanoate (PHA)-producing aerobic chemolithoautotrophy bacterium or a biopolymer-, renewable polymer- or biodegradable polymer-producing aerobic chemolithoautotrophy bacterium.

In alternative embodiments, of non-natural mixtures or consortium of bacteria as provided herein:
the PHA comprises polyhydroxybutyrate (PHB), or the biopolymer, renewable polymer or biodegradable polymer comprises: a polyhydroxyalkanoate (PHA): a poly(3-hydroxypropionate) (PHP or P3HP), a poly(3-hydroxybutyrate) (PHB or P3HB), a poly(4-hydroxybutyrate) (P4HB), a poly(3-hydroxyvalerate) (PHV or P3HV), a poly(4-hydroxyvalerate) (P4HV), a poly(5-hydroxyvalerate) (P5HV), a poly(3-hydroxyhexanoate) (PHHx or P3HHx), a poly(3-hydroxyoctanoate) (PHO, or P3HO), a poly(3-hydroxydecanoate) (PHD or P3HD), a poly(3-hydroxyundecanoate) (PHU, P3HU), a short- or medium-chain length, saturated or unsaturated PHA, a polylactic acid (PLA), or any copolymer thereof or any combination thereof;
the polyhydroxyalkanoate (PHA)-producing methanotrophic bacterium or biopolymer-, renewable polymer- or biodegradable polymer-producing methanotrophic bacterium are of or classified in (or derived from) the genus Methylobacterium, Methylosinus, Methylocella, Methylocapsa, Methylocaldum or Methylocystis,
and optionally the Methylocystis species is Methylocystis parvus,
and optionally the Methylosinus species is Methylosinus sporium,
and optionally the Methylocystis sp. is deposited under ATCC Accession Number ATCC 49242,
and optionally the Methylobacterium species is Methylobacterium extorquens,
and optionally the Methylobacterium extorquens is deposited under ATCC Accession Number ATCC 55366;
the polyhydroxyalkanoate (PHA)-producing methanotrophic bacterium or biopolymer-, renewable polymer- or biodegradable polymer-producing methanotrophic bacterium of or classified in (or derived from) the genus Methylocystis has a 16S ribosomal RNA (rRNA) sequence comprising at least about 90%, 95%, 96%, 97% or 98% or more, or complete (100%), sequence identity to a 16S rRNA sequence:

(SEQ ID NO: 1)
AACGAACGCTGGCGGCAGGCCTAACACATGCAAGTCGAACGCTGTAGCA
ATACAGAGTGGCAGACGGGTGAGTAACGCGTGGGAACGTGCCTTTCGGT
TCGGAATAACTCAGGGAAACTTGAGCTAATACCGGATACGCCCTTTGGG
GGAAAGATTTATTGCCGAAAGATCGGCCCGCGTCCGATTAGCTAGTTGG
TGTGGTAATGGCGCACCAAGGCGACGATCGGTAGCTGGTCTGAGAGGAT
GATCAGCCACACTGGGACTGAGACACGGCCCAGACTCCTACGGGAGGCA
GCAGTGGGGAATATTGGACAATGGGCGCAAGCCTGATCCAGCCATGCCG
CGTGAGTGATGAAGGCCCTAGGGTTGTAAAGCTCTTTCGCCAGGGACGA
TAATGACGGTACCTGGATAAGAAGCCCCGGCTAACTTCGTGCCAGCAGC
CGCGGTAATACGAAGGGGGCTAGCGTTGTTCGGAATCACTGGGCGTAAA
GCGCACGTAGGCGGATCTTTAAGTCAGGGGTGAAATCCCGAGGCTCAAC
CTCGGAACTGCCTTTGATACTGGAGGTCTCGAGTCCGGGAGAGGTGAGT
GGAACTGCGAGTGTAGAGGTGAAATTCGTAGATATTCGCAAGAACACCA
GTGGCGAAGGCGGCTCACTGGCCCGGTACTGACGCTGAGGTGCGAAAGC
GTGGGGAGCAAACAGGATTAGATACCCTGGTAGTCCACGCCGTAAACGA
TGGATGCTAGCCGTTGGGGAGCATGCTCTTCAGTGGCGCAGCTAACGCT
TTAAGCATCCCGCCTGGGGAGTACGGTCGCAAGATTAAAACTCAAAGGA
ATTGACGGGGGCCCGCACAAGCGGTGGAGCATGTGGTTTAATTCGAAGC
AACGCGCAGAACCTTACCAGCTTTTGACATGCCCGGTATGATCGCCAGA
GATGGCTTTCTTCCCGCAAGGGGCCGGAGCACAGGTGCTGCATGGCTGT
CGTCAGCTCGTGTCGTGAGATGTTGGGTTAAGTCCCGCAACGAGCGCAA
CCCTCGCCCTTAGTTGCCATCATTCAGTTGGGCACTCTAGGGGGACTGC
CGGTGATAAGCCGCGAGGAAGGTGGGGATGACGTCAAGTCCTCATGGCC
CTTACAGGCTGGGCTACACACGTGCTACAATGGCGGTGACAATGGGAAG
CGAAAGGGCGACCTGGAGCAAATCTCAAAAAGCCGTCTCAGTTCGGATT
GCACTCTGCAACTCGAGTGCATGAAGGTGGAATCGCTAGTAATCGCAGA
TCAGCACGCTGCGGTGAATACGTTCCCGGGCCTTGTACACACCGCCCGT
CACACCATGGGAGTTGGTTTTACCCGAAGGCGTTTCGCCAACCGCAAGG
AGGCAGGCGACCACGGTAGGGTCAGCGACTGGGGTG;

the polyhydroxyalkanoate (PHA)-producing methanotrophic bacterium or biopolymer-, renewable polymer- or biodegradable polymer-producing methanotrophic bacterium of or classified in (or derived from) the genus Methylosinus has a 16S ribosomal RNA (rRNA) sequence comprising at least about 90%, 95%, 96%, 97% or 98% or more, or complete (100%), sequence identity to a 16S rRNA sequence:

(SEQ ID NO: 2)
AACGAACGCTGGCGGCAGGCTTAACACATGCAAGTCGAACGGGCGCAGC
GATGCGTCAGTGGCAGACGGGTGAGTAACGCGTGGGAACGTACCTTTCG
GTTCGGAATAACTCAGGGAAACTTGAGCTAATACCGGATACGCCCTTAG
GGGGAAAGATTTATTGCCGAAAGATCGGCCCGCGTCCGATTAGCTAGTT
GGTGAGGTAAAGGCTCACCAAGGCGACGATCGGTAGCTGGTCTGAGAGG
ATGATCAGCCACACTGGGACTGAGACACGGCCCAGACTCCTACGGGAGG
CAGCAGTGGGGAATATTGGACAATGGGCGCAAGCCTGATCCAGCCATGC
CGCGTGAGTGATGAAGGCCCTAGGGTTGTAAAGCTCTTTCGCCAGGGAC
GATAATGACGGTACCTGGATAAGAAGCCCCGGCTAACTTCGTGCCAGCA
GCCGCGGTAATACGAAGGGGGCTAGCGTTGTTCGGAATCACTGGGCGTA
AAGCGCACGTAGGCGGATTGTTAAGTCAGGGGTGAAATCCCGAGGCTCA
ACCTCGGAACTGCCTTTGATACTGGCGATCTAGAGTCCGGGAGAGGTGA
GTGGAACTGCGAGTGTAGAGGTGAAATTCGTAGATATTCGCAAGAACAC
CAGTGGCGAAGGCGGCTCACTGGCCCGGAACTGACGCTGAGGTGCGAAA
GCGTGGGGAGCAAACAGGATTAGATACCCTGGTAGTCCACGCCGTAAAC
GATGGATGCTAGCCGTTGGGGAGCTTGCTCTTCAGTGGCGCAGCTAACG
CTTTAAGCATCCCGCCTGGGGAGTACGGTCGCAAGATTAAAACTCAAAG
GAATTGACGGGGGCCCGCACAAGCGGTGGAGCATGTGGTTTAATTCGAA
GCAACGCGCAGAACCTTACCAGCTTTTGACATGTCCGGTATGGTCGTCA
GAGATGACTTCCTTCCCGCAAGGGGCCGGAACACAGGTGCTGCATGGCT
GTCGTCAGCTCGTGTCGTGAGATGTTGGGTTAAGTCCCGCAACGAGCGC
AACCCTCGCCCTTAGTTGCCATCATTCAGTTGGGCACTCTAGGGGGACT
GCCGGTGATAAGCCGCGAGGAAGGTGGGGATGACGTCAAGTCCTCATGG
CCCTTACAGGCTGGGCTACACACGTGCTACAATGGCGGTGACAATGGGA
AGCGAAGGGGTGACCCGGAGCAAATCTCCAAAAGCCGTCTCAGTTCGGA
TTGCACTCTGCAACTCGAGTGCATGAAGGTGGAATCGCTAGTAATCGCA
GATCAGCACGCTGCGGTGAATACGTTCCCGGGCCTTGTACACACCGCCC
GTCACACCATGGGAGTTGGCTTTACCCGAAGGCGTTTCGCTAACCGCAA
GGAGGCAGACGACCACGGTAGGGTCAGCGACTGGGGTG;

the polyhydroxyalkanoate (PHA)-producing methanotrophic bacterium or biopolymer-, renewable polymer- or biodegradable polymer-producing methanotrophic bacterium of or classified in (or derived from) the genus Methylocaldum has a 16S ribosomal RNA (rRNA) sequence comprising at least about 90%, 95%, 96%, 97% or 98% or more, or complete (100%), sequence identity to a 16S rRNA sequence:

(SEQ ID NO: 3)
TTAAACTGAAGAGTTTGATCATGGCTCAGATTGAACGCTGGCGGCATGC
TTAACACATGCAAGTCGAACGGCAGCACAGCCGGGTAACCGGTGGGTGG
CGAGTGGCGGACGGGTGAGTAATGCGTAGGAATCTGCCTTGTAGTGGGG
GATAACTCGGGGAAACTCGGGCTAATACCGCATACGCTCTACGGAGGAA
AGCGGGGGATCTTCGGACCTCGCGCTATGAGATGAGCCTACGTCCGATT
AGCTAGTTGGCAGGGTAATGGCCTACCAAGGCGACGATCGGTAGCTGGT
CTGAGAGGACGATCAGCCACACTGGAACTGAGACACGGTCCAGACTCCT
ACGGGAGGCAGCAGTGGGGAATATTGGACAATGGGCGCAAGCCTGATCC
AGCAATGCCGCGTGTGTGAAGAAGGCCTGCGGGTTGTAAAGCACTTTAA
GCAGGGAAGAAAGGTGGGGTGTTAACACCATCTCACATTGACGTTACCT
GCAGAATAAGCACCGGCTAACTCCGTGCCAGCAGCCGCGGTAATACGGA
GGGTGCGAGCGTTAATCGGAATTACTGGGCGTAAAGCGCGCGTAGGCGG
TTTGTTAAGTCAGCTGTGAAAGCCCCGGGCTTAACCTGGGAATGGCAGT
TGATACTGGCGAGCTAGAGTGTGGTAGAGGGGTGTGGAATTTCCGGTGT
AGCAGTGAAATGCGTAGAGATCGGAAGGAACACCAGTGGCGAAGGCGGC
ACCCTGGACCAACACTGACGCTGAGGTGCGAAAGCGTGGGGAGCAAACA
GGATTAGATACCCTGGTAGTCCACGCTGTAAACGATGAGAACTAGCCGT
TGGGCACAATCGAGTGTTTAGTGGCGCAGCTAACGCGATAAGTTCTCCG
CCTGGGGAGTACGGCCGCAAGGTTAAAACTCAAATGAATTGACGGGGGC
CCGCACAAGCGGTGGAGCATGTGGTTTAATTCGATGCAACGCGAAGAAC
CTTACCTGGTCTTGACATGTCGCGAACCCTTGAGAGATCGAGGGGTGCC
TTCGGGAGCGCGAACACAGGTGCTGCATGGCTGTCGTCAGCTCGTGTCG
TGAGATGTTGGGTTAAGTCCCGTAACGAGCGCAACCCTTGTCCCTAGTT
GCCAGCGTTGAGTCGGGAACTCTAGGGAGACTGCCGGTGATAAACCGGA
GGAAGGTGGGGATGACGTCAAGTCATCATGGCCCTTATGACCAGGGCTA
CACACGTGCTACAATGGTCGGTACAGAGGGTAGCCAAGCCGTGAGGCGG
AGCCAATCTCACAAAGCCGATCGTAGTCCGGATTGCAGTCTGCAACTCG
ACTGCATGAAGTCGGAATCGCTAGTAATCGCGGATCAGCATGCCGCGGT
GAATACGTTCCCGGGCCTTGTACACACCGCCCGTCACACCATGGGAGTG
GGTTGCACCAGAAGCAGGTAGTCTAACCTTCGGGAGGGCGCTTGCCACG
GTGTGGTTCATGACTGGGGTGAAGTCGTAACAAGGTAGCCGTAGGGGAA
CCTGCGGCTGGATCACCTCCTTTCA;

the polyhydroxyalkanoate (PHA)-producing aerobic chemolithoautotrophy bacterium or the biopolymer-, renewable polymer- or biodegradable polymer-producing aerobic chemolithoautotrophy bacterium are of or classified in (or derived from) the genus Methyloversatilis, Rubrivivax, Rhodopseudomonas, Xanthobacter, Ralstonia, Cuprividus or Hydrogenophaga,
wherein optionally the Hydrogenophaga bacterium is an H. flava,
and optionally the Ralstonia is R. eutropha,
and optionally the Cuprividus is C. necator;
the polyhydroxyalkanoate (PHA)-producing aerobic chemolithoautotrophy bacterium or the biopolymer-, renewable polymer- or biodegradable polymer-producing aerobic chemolithoautotrophy bacterium of or classified in (or derived from) the genus Hydrogenophaga has a 16S ribosomal RNA (rRNA) sequence comprising at least about 90%, 95%, 96%, 97% or 98% or more, or complete (100%), sequence identity to a 16S rRNA sequence:

(SEQ ID NO:4)
AGTTTGATCCTGGCTCAGATTGAACGCTGGCGGCATGCTTTACACATGC
AAGTCGAACGGTAACAGGCCGCAAGGTGCTGACGAGTGGCGAACGGGTG
AGTAATGCATNGGAACGTGCCCAGTCGTGGGGGATAACGCAGCGAAAGC
TGTGCTAATACCGCATACGATCTATGGATGAAAGCGGGGGACCGTAAGG
CCTCGCGCGATTGGAGCGGCCGATGTCAGATTAGATAGTTGGTGGGGTA
AAGGCTCACCAAGCCAACGATCTGTAGCTGGTCTGAGAGGACGGCCAGC
CACACTGGGACTGAGACACGGCCCAGACTCCTACGGGAGGCAGCAGTGG
GGAATTTTGGACAATGGGCGCAAGCCTGATCCAGCAATGCCGCGTGCAG
GAAGAAGGCCTTCGGGTTGTAAACTGCTTTTGTACGGAACGAACGGTCT
TGGGTTAATACCTCGGGCTAATGACGGTACCGTAAGAATAAGCACCGGC
TAACTACGTGCCAGCAGCCGCGGTAATACGTAGGGTGCAAGCGTTAATC
GGAATTACTGGGCGTAAAGCGTGCGCAGGCGGTTTTGTAAGACAGGCGT
GAAATCCCCGGGCTCAACCTGGGAATTGCGCTTGTNACTGCAAGGCTGG
AGTGCGGCAGAGGGGGATGGAATTCCGCGTGTAGCAGTGAAATGCGTAG
ATATGCGGAGGAACACCGATGGCGAAGGCAATCCCCTGGGCCTGCACTG
ACGCTCATGCACGAAAGCGTGGGGAGCAAACAGGATTAGATACCCTGGT
AGTCCACGCCCTAAACGATGTCAACTGGTTGTTGGGAATTTACCTTCTC
AGTAACGAAGCTAACGCGTGAAGTTGACCGCCTGGGGAGTACGGCCGCA
AGGTTGAAACTCAAAGGAATTGACGGGGACCCGCACAAGCGGTGGATGA
TGTGGTTTAATTCGATGCAACGCGAAAAACCTTACCCACCTTTGACATG
GCAGGAAGTTTCCAGAGATGGATTCGTGCTCGAAAGAGAACCTGCACAC
AGGTGCTGCATGGCTGTCGTCAGCTCGTGTCGTGAGATGTTGGGTTAAG
TCCCGCAACGAGCGCAACCCTTGCCATTAGTTGCTACGAAAGGGCACTC
TAATGNGACTGCCGGTGACAAACCGGAGGAAGGTGGGGATGACGTCAAG
TCCTCATGGCCCTTATAGGTGGGGCTACACACGTCATACAATGGCCGGT
ACAAAGGGCAGCCAACCCGCGAGGGGGAGCCAATCCCATAAAGCCGGTC
GTAGTCCGGTTCGCAGTCTGCAACTCGACTGCGTGAAGTCGGAATCGCT
AGTAATCGTGGATCAGCATGTCACGGTGAATACGTTCCCGGGTCTTGTA
CACACCGCCCGTCACACCATGGGAGCGGGTCTCGCCAGAAGTAGTTAGC
CTAACCGCAAGGAGGGCGATTACCACGGCGGGGTTCGTGACTGGGGTGA
AGTCGTAACAAGGTAGCCGTATCGGAAGGTGCGGCTGGATCACCTCCT
T;

the polyhydroxyalkanoate (PHA)-producing aerobic chemolithoautotrophy bacterium or the biopolymer-, renewable polymer- or biodegradable polymer-producing aerobic chemolithoautotrophy bacterium of or classified in (or derived from) the genus Xanthobacter has a 16S ribosomal RNA (rRNA) sequence comprising at least about 90%, 95%, 96%, 97% or 98% or more, or complete (100%), sequence identity to a 16S rRNA sequence:

(SEQ ID NO: 5)
AGCGAACGCTGGCGGCAGGCCTAACACATGCAAGTCGAGCGCCCAGCAA
TGGGAGCGGCAGACGGGTGAGTAACACGTGGGGATCTACCCAATGGTAC
GGAATAACCCAGGGAAACTTGGACTAATACCGTATGTGCCCTTCGGGGG
AAAGATTTATCGCCATTGGATGAACCCGCGTCGGATTAGCTAGTTGGTG
AGGTAAAGGCTCACCAAGGCGACGATCCGTAGCTGGTCTGAGAGGATGA
TCAGCCACACTGGGACTGAGACACGGCCCAGACTCCTACGGGAGGCAGC
AGTGGGGAATATTGGACAATGGGCGCAAGCCTGATCCAGCCATGCCGCG
TGTGTGATGAAGGCCTTAGGGTTGTAAAGCACTTTCGCCGGTGAAGATA
ATGACGGTAACCGGAGAAGAAGCCCCGGCTAACTTCGTGCCAGCAGCCG
CGGTAATACGAAGGGGGCAAGCGTTGCTCGGAATCACTGGGCGTAAAGC
GCACGTAGGCGGGTTGTTAAGTCAGAGGTGAAATCCTGGAGCTCAACTC
CAGAACTGCCTTTGATACTGGCGACCTTGAGTTCGAGAGAGGTTGGTGG
AACTGCGAGTGTAGAGGTGAAATTCGTAGATATTCGCAAGAACACCAGT
GGCGAAGGCGGCCAACTGGCTCGATACTGACGCTGAGGTGCGAAAGCGT
GGGGAGCAAACAGGATTAGATACCCTGGTAGTCCACGCCGTAAACGATG
GATGCTAGCCGTTAGGCAGCTTGCTGCTTAGTGGCGCAGCTAACGCATT
AAGCATCCCGCCTGGGGAGTACGGTCGCAAGATTAAAACTCAAAGGAAT
TGACGGGGGCCCGCACAAGCGGTGGAGCATGTGGTTTAATTCGAAGCAA
CGCGCAGAACCTTACCAGCCTTTGACATGGCAGGACGATTTCCAGAGAT
GGATCTCTTCCAGCAATGGACCTGCACACAGGTGCTGCATGGCTGTCGT
CAGCTCGTGTCGTGAGATGTTGGGTTAAGTCCCGCAACGAGCGCAACCC
TCGCCTCTAGTTGCCAGCATTCAGTTGGGCACTCTAGAGGGACTGCCGG
TGATAAGCCGCGAGGAAGGTGGGGATGACGTCAAGTCCTCATGGCCCTT
ACGGGCTGGGCTACACACGTGCTACAATGGCGGTGACAGTGGGATGCAA
AGGGGCGACCCCTAGCAAATCTCCAAAAGCCGTCTCAGTTCGGATTGCA
CTCTGCAACTCGAGTGCATGAAGTTGGAATCGCTAGTAATCGTGGATCA
GCATGCCACGGTGAATACGTTCCCGGGCCTTGTACACACCGCCCGTCAC
ACCATGGGAGTTGGCTTTACCCGAAGGCGCTGCGCTAACCCGCAAGGGA
GGCAGGCGACCACGGTAGGGTCAGCGACTGGGGTG;

the polyhydroxyalkanoate (PHA)-producing aerobic chemolithoautotrophy bacterium or the biopolymer-, renewable polymer- or biodegradable polymer-producing aerobic chemolithoautotrophy bacterium of or classified in (or derived from) the genus Ralstonia or Cuprividus has a 16S ribosomal RNA (rRNA) sequence comprising at least about 90%, 95%, 96%, 97% or 98% or more, or complete (100%), sequence identity to a 16S rRNA sequence:

(SEQ ID NO: 6)
AGAGTTTGATCCTGGCTCAGATTGAACGCTGGCGGCATGCCTTACACAT
GCAAGTCGAACGGCAGCACGGGCTTCGGCCTGGTGGCGAGTGGCGAACG
GGTGAGTAATACATCGGAACGTGCCCTGTAGTGGGGGATAACTAGTCGA
AAGATTAGCTAATACCGCATACGACCTGAGGGTGAAAGCGGGGGACCGA
AGGCCTCGCGCTACAGGAGCGGCCGATGTCTGATTAGCTAGTTGGTGGG
GTAAAGCCTACCAAGGCGACGATCAGTAGCTGGTCTGAGAGGACGATCA
GCCACACTGGGACTGAGAACACGGCCCAGACTCCTACGGAGGCAGCAGT
GGGGAATTTTGGACAATGGGGGCAACCCTGATCCAGCAATGCCGCGTGT
GTGAAGAAGGCCTTCGGGTTTGTAAAGCACTTTTGTCCGGAAAGAAATG
GCTCTGGTTAATACCCGGGGTCGATGACGGTACCGGAAGAATAAGCACC
GGCTAACTACGTGCCAGCAGCCGCGGTAATACGTAGGGTGCGAGCGTTA
ATCGGAATTACTGGGCGTAAAGCGTGCGCAGGCGGTTTTGTAAGACAGG
CGTGAAATCCCCGAGCTCAACTTGGGAATGGCGCTTGTGACTGCAAGGC
TAGAGTATGTCAGAGGGGGGTAGAATTCCACGTGTAGCAGTGAAATGCG
TAGAGATGTGGAGGAATACCGATGGCGAAGGCAGCCCCCTGGGACGTCA
CTGACGCTCATGCACGAAAGCGTGGGGAGCAAACAGGATTAGATACCCT
GGTAGTCCACGCCCTAAACGATGTCAACTAGTTGTTGGGGATTCATTTC
TTCAGTAACGTAGCTAACGCGTGAAGTTGACCGCCTGGGGAGTACGGTC
GCAAGATTAAAACTCAAAGGAATTGACGGGGACCGCACAAGCGGTGGAT
GATGTGGATTAATTCGATGCAACGCGAAAAACCTTACCTACCCTTGACA
TGCCACTAACGAAGCAGAGATGCATTAGGTGTCCGAAAGGGAGAGTGGA
CACAGGTGCTGCATGGCTGTCGTCAGCTCGTGTCGTGAGATGTTGGGTT
AAGTCCCGCAACGAGCGCAACCCTTGTCTCTAGTTGCTACGAAAGGGCA
CTCTAGAGAGACTGCCGGTGACAAACCGGAGGAAGGTGGGGATGACGTC
AAGTCCTCATGGCCTTATGGGTAGGGCTTCACACGTCATACAATGGTGC
GTACAGAGGGTTGCCAACCCGCGAGGGGGAGCTAATCCCAGAAAACGCA
TCGTAGTCCGGATCGTAGTCTGCAACTCGACTACGTGAAGCTGGAATCG
CTAGTAATCGCGGATCAGCATGCCGCGGTGAATACGTTCCCGGGTCTTG
TACACACCGCCCGTCACACCATGGGAGTGGGTTTTGCCAGAAGTAGTTA
GCCTAACCGCAAGGAGGGCGATTACCACAGCAGGGTTCATGACTGGGGT
GAAGTCGTAACAAGGTAACC;

the polyhydroxyalkanoate (PHA)-producing aerobic chemolithoautotrophy bacterium or the biopolymer-, renewable polymer- or biodegradable polymer-producing aerobic chemolithoautotrophy bacterium of or classified in (or derived from) the genus Rubrivivax has a 16S ribosomal RNA (rRNA) sequence comprising at least about 90%, 95%, 96%, 97% or 98% or more, or complete (100%), sequence identity to a 16S rRNA sequence:

(SEQ ID NO: 7)
TCAGATTGAACGCTGGCGGCATGCCTTACACATGCAAGTCGAACGGTAA
CAGGCCGCAAGGTGCTGACGAGTGGCGAACGGGTGAGTAATGCATCGGA
ACGTGCCCAGTAGTGGGGGATAGCCCGGCGAAAGCCGGATTAATACCGC
ATACGACCTATGGGTGAAAGCGGGGGACCGAAAGGCCTCGCGCTATTGG
AGCGGCCGATGTCAGATTAGGTAGTTGGTGGGGTAAAGGCCTACCAAGC
CTACGATCTGTAGCTGGTCTGAGAGGACGACCAGCCACACTGGGACTGA
GACACGGCCCAGACTCCTACGGGAGGCAGCAGTGGGGAATTTTGGACAA
TGGGCGCAAGCCTGATCCAGCCATGCCGCGTGCGGGAAGAAGGCCTTCG
GGTTGTAAACCGCTTTTGTCAGGGAAGAAATCTTCTGGGTTAATACCTC
GGGAGGATGACGGTACCTGAAGAATAAGCACCGGCTAACTACGTGCCAG
CAGCCGCGGTAATACGTAGGGTGCAAGCGTTAATCGGAATTACTGGGCG
TAAAGCGTGCGCAGGCGGTTATGTAAGACAGATGTGAAATCCCCGGGCT
TAACCTGGGAACTGCATTTGTGACTGCATAGCTTGAGTGCGGCAGAGGG
GGATGGAATTCCGCGTGTAGCAGTGAAATGCGTAGATATGCGGAGGAAC
ACCGATGGCGAAGGCAATCCCCTGGGCCTGCACTGACGCTCATGCACGA
AAGCGTGGGGAGCAAACAGGATTAGATACCCTGGTAGTCCACGCCCTAA
ACGATGTCAACTGGTTGTTGGGAGGGTTTCTTCTCAGTAACGTAGCTAA
CGCGTGAAGTTGACCGCCTGGGGAGTACGGCCGCAAGGTTGAAACTCAA
AGGAATTGACGGGGACCCGCACAAGCGGTGGATGATGTGGTTTAATTCG
ACGCAACGCGAAAAACCTTACCTACCCTTGACATGCCAGGGATCCTGCA
GAGATGTGGGAGTGCTCGAAAGAGAACCTGGACACAGGTGCTGCATGGC
CGTCGTCAGCTCGTGTCGTGAGATGTTGGGTTAAGTCCCGCAACGAGCG
CAACCCTTGTCATTAGTTGCTACGTAAGGGCACTCTAATGAGACTGCCG
GTGACAAACCGGAGGAAGGTGGGGATGACGTCAGGTCATCATGGCCCTT
ATGGGTAGGGCTACACACGTCATACAATGGCCGGTACAGAGGGCTGCCA
ACCCGCGAGGGGGAGCCAATCCCAGAAAACCGGTCGTAGTCCGGATCGC
AGTCTGCAACTCGACTGCGTGAAGTCGGAATCGCTAGTAATCGCGGATC
AGCTTGCCGCGGTGAATACGTTCCCGGGTCTTGTACACACCGCCCGTCA
CACCATGGGAGCGGGTTCTGCCAGAAGTAGTTAGCCTAACCGCAAGGAG
GGCGATTACCACGGCAGGGTTCGTGACTGGGGTGAAGTCGTAACAAGGT
A;

the polyhydroxyalkanoate (PHA)-producing aerobic chemolithoautotrophy bacterium or the biopolymer-, renewable polymer- or biodegradable polymer-producing aerobic chemolithoautotrophy bacterium of or classified in (or derived from) the genus Methyloversatilis has a 16S ribosomal RNA (rRNA) sequence comprising at least about 90%, 95%, 96%, 97% or 98% or more, or complete (100%), sequence identity to a 16S rRNA sequence:

(SEQ ID NO: 8)
AGAGTTTGATTATGGCTCAGATTGAACGCTGGCGGAATGCTTTACACAT
GCAAGTCGAGCGGCAGCACGGGGGTAACCCTGGTGGCGAGCGGCGAACG
GGTGAGTAATACATCGGAACATGCCCAGTCGTGGGGGATAACACTTCGA
AAGAAGTGCTAATACCGCATACGTCCTGAGGGAGAAAGCGGGGGATCGC
AAGACCTCGCGCGATTGGAGTGGCCGATGTCAGATTAGCTAGTTGGTGG
GGTAAAGGCCTACCAAGGCAACGATCTGTAGCTGGTCTGAGAGGATGAT
CAGCCACACTGGGACTGAGACACGGCCCAGACTCCTACGGGAGGCAGCA
GTGGGGAATTTTGGACAATGGGGGCAACCCTGATCCAGCCATTCCGCGT
GAGTGAAGAAGGCCTTCGGGTTGTAAAGCTCTTTCGGCAGGAACGAAAC
GGTGAGCTCTAACATAGCTTGCTAATGACGGTACCTGAAGAAGAAGCAC
CGGCTAACTACGTGCCAGCAGCCGCGGTAATACGTAGGGTGCGAGCGTT
AATCGGAATTACTGGGCGTAAAGCGTGCGCAGGCGGTTGTGTAAGACAG
GTGTGAAATCCCCGGGCTTAACCTGGGAACTGCGCTTGTGACTGCACAG
CTAGAGTATGGCAGAGGGGGGTGGAATTCCACGTGTAGCAGTGAAATGC
GTAGAGATGTGGAGGAACACCGATGGCGAAGGCAGCCCCCTGGGCCAAT
ACTGACGCTCATGCACGAAAGCGTGGGGAGCAAACAGGATTAGATACCC
TGGTAGTCCACGCCCTAAACGATGTCGACTAGGTGTTCGGTGAGGAGAC
TCATTGAGTACCGCAGCTAACGCGTGAAGTCGACCGCCTGGGGAGTACG
GTCGCAAGATTAAAACTCAAAGGAATTGACGGGGACCCGCACAAGCGGT
GGATGATGTGGATTAATTCGATGCAACGCGAAAAACCTTACCTACCCTT
GACATGTACGGAACCCTGCTGAGAGGTGGGGGTGCTCGAAAGAGAGCCG
TAACACAGGTGCTGCATGGCTGTCGTCAGCTCGTGTCGTGAGATGTTGG
GTTAAGTCCCGCAACGAGCGCAACCCTTGTCATTAGTTGCTACGCAAGA
GCACTCTAATGAGACTGCCGGTGACAAACCGGAGGAAGGTGGGGATGAC
GTCAAGTCCTCATGGCCCTTATGGGTAGGGCTTCACACGTCATACAATG
GTCGGTACAGAGGGTTGCCAAGCCGCGAGGTGGAGCCAATCCCAGAAAG
CCGATCGTAGTCCGGATCGCAGTCTGCAACTCGACTGCGTGAAGTCGGA
ATCGCTAGTAATCGCGGATCAGCATGCCGCGGTGAATACGTTCCCGGGT
CTTGCACACACCGCCCGTC;

the polyhydroxyalkanoate (PHA)-producing aerobic chemolithoautotrophy bacterium or the biopolymer-, renewable polymer- or biodegradable polymer-producing aerobic chemolithoautotrophy bacterium of or classified in (or derived from) the genus Rhodopseudomonas has a 16S ribosomal RNA (rRNA) sequence comprising at least about 90%, 95%, 96%, 97% or 98% or more, or complete (100%), sequence identity to a 16S rRNA sequence:

(SEQ ID NO: 9)
AGCGAACGCTGGCGGCAGGCTTAACACATGCAAGTCGAACGGGCGTAGC
AATACGTCAGTGGCAGACGGGTGAGTAACGCGTGGGAACGTACCTTTTG
GTTCGGAACAACACAGGGAAACTTGTGCTAATACCGGATAAGCCCTTAC
GGGGAAAGATTTATCGCCGAAAGATCGGCCCGCGTCTGATTAGCTAGTT
GGTGAGGTAATGGCTCACCAAGGCGACGATCAGTAGCTGGTCTGAGAGG
ATGATCAGCCACATTGGGACTGAGACACGGCCCAAACTCCTACGGGAGG
CAGCAGTGGGGAATATTGGACAATGGGGGAAACCCTGATCCAGCCATGC
CGCGTGAGTGATGAAGGCCCTAGGGTTGTAAAGCTCTTTTGTGCGGGAA
GATAATGACGGTACCGCAAGAATAAGCCCCGGCTAACTTCGTGCCAGCA
GCCGCGGTAATACGAAGGGGGCTAGCGTTGCTCGGAATCACTGGGCGTA
AAGGGTGCGTAGGCGGGTTTCTAAGTCAGAGGTGAAAGCCTGGAGCTCA
ACTCCAGAACTGCCTTTGATACTGGAAGTCTTGAGTATGGCAGAGGTGA
GTGGAACTGCGAGTGTAGAGGTGAAATTCGTAGATATTCGCAAGAACAC
CAGTGGCGAAGGCGGCTCACTGGGCCATTACTGACGCTGAGGCACGAAA
GCGTGGGGAGCAAACAGGATTAGATACCCTGGTAGTCCACGCCGTAAAC
GATGAATGCCAGCCGTTAGTGGGTTTACTCACTAGTGGCGCAGCTAACG
CTTTAAGCATTCCGCCTGGGGAGTACGGTCGCAAGATTAAAACTCAAAG
GAATTGACGGGGGCCCGCACAAGCGGTGGAGCATGTGGTTTAATTCGAC
GCAACGCGCAGAACCTTACCAGCCCTTGACATGTCCAGGACCGGTCGCA
GAGACGCGACCTTCTCTTCGGAGCCTGGAGCACAGGTGCTGCATGGCTG
TCGTCAGCTCGTGTCGTGAGATGTTGGGTTAAGTCCCGCAACGAGCGCA
ACCCCCGTCCTTAGTTGCTACCATTTAGTTGAGCACTCTAAGGAGACTG
CCGGTGATAAGCCGCGAGGAAGGTGGGGATGACGTCAAGTCCTCATGGC
CCTTACGGGCTGGGCTACACACGTGCTACAATGGCGGTGACAATGGGAA
GCTAAGGGGCGACCCTTCGCAAATCTCAAAAAGCCGTCTCAGTTCGGAT
TGGGCTCTGCAACTCGAGCCCATGAAGTTGGAATCGCTAGTAATCGTGG
ATCAGCATGCCACGGTGAATACGTTCCCGGGCCTTGTACACACCGCCCG
TCACACCATGGGAGTTGGCTTTACCTGAAGACGGTGCGCTAACCAGCAA
GTGGGGGCAGCCGGCCACGGTAGGGTCAGCGACTGGGGTG;

the non-natural mixture or consortium of bacteria comprises:
   at least one bacterium from the genus Methylobacterium and at least one bacterium from the genus Methyloversatilis;
   at least one bacterium from the genus Methylobacterium and at least one bacterium from the genus Rubrivivax;
   at least one bacterium from the genus Methylobacterium and at least one bacterium from the genus Rhodopseudomonas;
   at least one bacterium from the genus Methylobacterium and at least one bacterium from the genus Xanthobacter;
   at least one bacterium from the genus Methylobacterium and at least one bacterium from the genus Ralstonia;
   at least one bacterium from the genus Methylobacterium and at least one bacterium from the genus Cuprividus; or
   at least one bacterium from the genus Methylobacterium and at least one bacterium from the genus Hydrogenophaga;
   at least one bacterium from the genus Methylosinus and at least one bacterium from the genus Methyloversatilis;
   at least one bacterium from the genus Methylosinus and at least one bacterium from the genus Rubrivivax;
   at least one bacterium from the genus Methylosinus and at least one bacterium from the genus Rhodopseudomonas;
   at least one bacterium from the genus Methylosinus and at least one bacterium from the genus Xanthobacter;
   at least one bacterium from the genus Methylosinus and at least one bacterium from the genus Ralstonia;
   at least one bacterium from the genus Methylosinus and at least one bacterium from the genus Cuprividus; or
   at least one bacterium from the genus Methylosinus and at least one bacterium from the genus Hydrogenophaga;
   at least one bacterium from the genus Methylocella and at least one bacterium from the genus Methyloversatilis;
   at least one bacterium from the genus Methylocella and at least one bacterium from the genus Rubrivivax;
   at least one bacterium from the genus Methylocella and at least one bacterium from the genus Rhodopseudomonas;
   at least one bacterium from the genus Methylocella and at least one bacterium from the genus Xanthobacter;
   at least one bacterium from the genus Methylocella and at least one bacterium from the genus Ralstonia;
   at least one bacterium from the genus Methylocella and at least one bacterium from the genus Cuprividus; or
   at least one bacterium from the genus Methylocella and at least one bacterium from the genus Hydrogenophaga;
   at least one bacterium from the genus Methylocella and at least one bacterium from the genus Methyloversatilis;
   at least one bacterium from the genus Methylocapsa and at least one bacterium from the genus Rubrivivax;
   at least one bacterium from the genus Methylocapsa and at least one bacterium from the genus Rhodopseudomonas;
   at least one bacterium from the genus Methylocapsa and at least one bacterium from the genus Xanthobacter;
   at least one bacterium from the genus Methylocapsa and at least one bacterium from the genus Ralstonia;
   at least one bacterium from the genus Methylocapsa and at least one bacterium from the genus Cuprividus; or
   at least one bacterium from the genus Methylocapsa and at least one bacterium from the genus Hydrogenophaga;
   at least one bacterium from the genus Methylocaldum and at least one bacterium from the genus Methyloversatilis;
   at least one bacterium from the genus Methylocaldum and at least one bacterium from the genus Rubrivivax;
   at least one bacterium from the genus Methylocaldum and at least one bacterium from the genus Rhodopseudomonas;
   at least one bacterium from the genus Methylocaldum and at least one bacterium from the genus Xanthobacter;
   at least one bacterium from the genus Methylocaldum and at least one bacterium from the genus Ralstonia;
   at least one bacterium from the genus Methylocaldum and at least one bacterium from the genus Cuprividus; or
   at least one bacterium from the genus Methylocaldum and at least one bacterium from the genus Hydrogenophaga;
   at least one bacterium from the genus Methylocystis and at least one bacterium from the genus Methyloversatilis;
   at least one bacterium from the genus Methylocystis and at least one bacterium from the genus Rubrivivax;
   at least one bacterium from the genus Methylocystis and at least one bacterium from the genus Rhodopseudomonas;
   at least one bacterium from the genus Methylocystis and at least one bacterium from the genus Xanthobacter;
   at least one bacterium from the genus Methylocystis and at least one bacterium from the genus Ralstonia;
   at least one bacterium from the genus Methylocystis and at least one bacterium from the genus Cuprividus; or
   at least one bacterium from the genus Methylocystis and at least one bacterium from the genus Hydrogenophaga.

In alternative embodiments, provided are genetically engineered bacterium, wherein the bacterium is or is derived from a bacterium in a mixture or consortium as provided herein, having contained therein at least one heterologous nucleic acid encoding an enzyme involved in biopolymer, renewable polymer or biodegradable polymer, or polyhydroxyalkanoate (PHA) synthesis.

In alternative embodiments, the bacteria in the non-natural mixtures or consortium of bacteria as provided herein are genetically engineered, for example:
the polyhydroxyalkanoate (PHA)-producing methanotrophic bacterium or biopolymer-, renewable polymer- or biodegradable polymer-producing methanotrophic bacterium, are genetically engineered, and/or
the polyhydroxyalkanoate (PHA)-producing aerobic chemolithoautotrophy bacterium or a biopolymer-, renewable polymer- or biodegradable polymer-producing aerobic chemolithoautotrophy bacterium are genetically engineered.

In alternative embodiments of the genetically engineered bacterium at least one heterologous nucleic acid encoding an enzyme (involved in biopolymer, renewable polymer or biodegradable polymer, or polyhydroxyalkanoate (PHA) synthesis) is implanted transiently or stably into the bacterium, for example:
the at least one heterologous nucleic acid encodes a 0-ketothiolase or an acetoacetyl-CoA reductase, for example:
the β-ketothiolase nucleic acid is derived from a Methylobacterium sp. strain 1805 beta-ketothiolase (phaA) gene, for example as set forth in GenBank: KY229163.1; or
the genetically engineered bacterium have contained therein a heterologous operon for polyhydroxyalkanoate (PHA) biosynthesis; or
the genetically engineered bacterium have contained therein polyhydroxybutyrate (PHB) synthesis genes (phbCAB) or a phbCAB operon, including beta-Ketothiolase (PhbA), acetoacetyl-CoA reductase (PhbB) and/or polyhydroxyalkanoate (PHA) synthase (PhbC) coding sequences, as described for example, by Zhang et al, Appl Microbiol Biotechnol. 2006 June; 71(2): 222-7.

In alternative embodiments of non-natural mixture or consortium of bacteria as provided herein:
(a) at least one polyhydroxyalkanoate (PHA)-producing methanotrophic bacterium or biopolymer-, renewable polymer- or biodegradable polymer-producing methanotrophic bacterium has contained therein at least one heterologous nucleic acid encoding an enzyme involved in biopolymer, renewable polymer or biodegradable polymer or polyhydroxyalkanoate (PHA) synthesis; or
(b) at least one polyhydroxyalkanoate (PHA)-producing aerobic chemolithoautotrophy bacterium or a biopolymer-, renewable polymer- or biodegradable polymer-producing aerobic chemolithoautotrophy bacterium has contained therein at least one heterologous nucleic acid encoding an enzyme involved in biopolymer, renewable polymer or biodegradable polymer or polyhydroxyalkanoate (PHA) synthesis.

In alternative embodiments, provided are products of manufacture comprising a non-natural mixture or a consortium of bacteria as provided herein.

In alternative embodiments of products of manufacture as provided herein:
the product of manufacture is fabricated or constructed as a bioreactor;
the bacterium or the non-natural mixture or consortium of bacteria are attached to or contained in a plurality of macro- or nano-particles, microfibers, microtubes, microribbons and/or microbeads, or are enclosed in or immobilized in or onto a hydrogel, a crystal gel matrix or a nanoshell, or equivalent,
wherein optionally the plurality of macro- or nano-particles, microfibers, microtubes, microribbons and/or microbeads are arranged or fabricated as an array or a sheet,
and optionally the bacterium-comprising crystal gel matrix or a nanoshell or equivalent, are attached to or immobilized on to the plurality of macro- or nano-particles, microfibers, microtubes, microribbons and/or microbeads, or the bacterium-comprising crystal gel matrix or a nanoshell or equivalent, are attached to or immobilized on to a mesh or equivalent supporting structure;
the bacterium are encapsulated or enclosed or immobilized in polymer, a colloidal particle shell, a dendrimer, an agar or a gel, or a hydrogel,
and optionally the encapsulated structures are immobilized onto the arrays or sheets, macro- or nano-particles, microfibers, microtubes, microribbons and/or microbeads;
the arrays, sheets, macro- or nano-particles, microfibers, microtubes, microribbons and/or microbeads are contained in or fabricated as sheets, mats, meshes, cartridges or any form of secondary or tertiary structure to support the arrays, macro- or nano-particles, microfibers, microtubes, microribbons and/or microbeads;
the arrays, macro- or nano-particles, microfibers, microtubes, microribbons and/or microbeads, or sheets, meshes, mats, cartridges or any form of secondary structure, are fabricated into modular units or cartridges that can be inserted into a superstructure or device,
and optionally the modular units or cartridges are fabricated to be exchanged or inserted into a preformed receptacle in or on the superstructure or device,
and optionally the modular units or cartridges or equivalent structures are fabricated to have gas input and output openings or orifices,
and optionally the superstructure or device comprises a pump, valves and/or pressure gauges controlling the amount of air or gas flow into or through the product of manufacture; and/or
the bacterium or the non-natural mixture or consortium of bacteria, or the arrays, macro- or nano-particles, microfibers, microtubes, microribbons and/or microbeads, or sheets, meshes, mats, cartridges or any form of secondary structure, or modular units or cartridges, or superstructures or devices, are fabricated as or are incorporated or integrated within a bioreactor.

In alternative embodiments, provided are methods for making a biopolymer, renewable polymer or biodegradable polymer,
wherein optionally the biopolymer, renewable polymer or biodegradable polymer comprises: a polyhydroxyalkanoate (PHA), and optionally the PHA comprises a polyhydroxybutyrate (PHB), comprising:
(a) culturing a non-natural mixture or consortium of bacteria as set forth herein, or culturing a non-natural mixture or consortium of bacteria in or contained in or on a product of manufacture as provided herein; and
(b) isolating, separating, purifying or harvesting the biopolymer, renewable polymer or biodegradable polymer.

In alternative embodiments of methods as provided herein:
the bacterium or a mixture or consortium of bacteria are cultured under conditions comprising adding to the culture: methane (CH₄) and air; methane (CH₄) and oxygen (O₂); methane (CH₄), hydrogen and air; methane (CH₄) and hydrogen, and/or air; methane (CH₄), carbon dioxide (CO₂); methane (CH₄) and carbon dioxide (CO₂) and air; methane (CH₄) carbon dioxide (CO₂) and oxygen (O₂); methane (CH₄), carbon dioxide (CO₂) and hydrogen; and/or, methane (CH₄), carbon dioxide (CO₂), air and/or oxygen (O₂), and hydrogen, and optionally reagents comprising methanol, acetate, formate and/or succinate are also added to the culture;
in alternative embodiments, the methane (CH₄) are added to the culture in the form of natural gas and/or biogas;
in alternative embodiments, the culture conditions comprise use of a closed container or reaction vessel, and optionally the methane (CH₄), carbon dioxide (CO₂), air and/or oxygen (O₂), and hydrogen are added as gases, and optionally one or all of these gases are added under pressure into the closed container or reaction vessel;
the bacterium or a mixture or consortium of bacteria are cultured under conditions comprising a temperature of between about 20° C. to 37° C. or between about 15° C. to 40° C.;
the biopolymer, renewable polymer or biodegradable polymer is separated, isolated or purified,
and optionally the biopolymer, renewable polymer or biodegradable polymer is separated, isolated or purified by a method or process comprising: decantation, filtration, centrifugation, aggregation, air flotation or precipitation or any combinations thereof;
and optionally the biopolymer, renewable polymer or biodegradable polymer is separated, isolated or purified to between about 70% to 99.5% purity, or at least about 80%, 85%, 90%, 95%, 97%, or 99% purity,
and optionally the biopolymer, renewable polymer or biodegradable polymer is separated, isolated or purified by a method or process comprising use of a process as described in U.S. patent application publication no. US 2017 0253713 A1, or U.S. patent no. (USPN) 10,597,506 or U.S. Pat. No. 8,852,157;
the biopolymer, renewable polymer or biodegradable polymer is separated, isolated or purified by a method or process comprising a lysis step, and optionally the lysis step comprises lysing a bacterial cell, and optionally the lysing of a bacterial cell comprises use of one or more extracellular enzymes;
the biopolymer, renewable polymer or biodegradable polymer is separated, isolated or purified by a method or process further comprising a separation or extraction step, wherein proteins, peptides, or amino acids or any combinations thereof are separated or extraction from the biopolymer, renewable polymer or biodegradable polymer;
the methods further comprise a washing step, and optionally the washing is after the extraction, isolation and/or separation to wash an obtained biopolymer, renewable polymer or biodegradable polymer, wherein optionally the washing step is performed using a reagent comprising water as a washing liquid;
no organic solvent or chemicals or any combinations thereof are used in the extraction, separation or isolation process;
the biopolymer, renewable polymer or biodegradable polymer comprises a poly-4-hydroxybutyrate (P4HB) and/or a copolymer thereof;
the biopolymer, renewable polymer or biodegradable polymer comprises: a polyhydroxyalkanoate (PHA): a poly(3-hydroxypropionate) (PHP or P3HP), a poly(3-hydroxybutyrate) (PHB or P3HB), a poly(4-hydroxybutyrate) (P4HB), a poly(3-hydroxyvalerate) (PHV or P3HV), a poly(4-hydroxyvalerate) (P4HV), a poly(5-hydroxyvalerate) (P5HV), a poly(3-hydroxyhexanoate) (PHHx or P3HHx), a poly(3-hydroxyoctanoate) (PHO, or P3HO), a poly(3-hydroxydecanoate) (PHD or P3HD), a poly(3-hydroxyundecanoate) (PHU, P3HU), a short- or medium-chain length, saturated or unsaturated PHA, a polylactic acid (PLA), or any copolymer thereof or any combination thereof; and/or
the bacterium or the mixture or consortium of bacteria produce or generate in culture at least 5 grams (g) L⁻¹ h⁻¹ PHA.

In alternative embodiments, provided are processes for producing a monomer from a biopolymer, renewable polymer or biodegradable polymer, comprising:
(a) providing a biopolymer, renewable polymer or biodegradable polymer produced, isolated or separated using a method as provided herein,
(b) providing a depolymerization substance or reagent; and
(c) depolymerizing the biopolymer, renewable polymer or biodegradable polymer by admixing the biopolymer, renewable polymer or biodegradable polymer and the depolymerization substance,
wherein optionally the depolymerization substance or reagent comprises a thermostable extracellular PHA depolymerase.

In alternative embodiments, provided are methods of making a biocompatible product, a bioactive nanobead, a biodegradable, disposable or renewable product, a thermoplast, an elastomer product or a polymer precursor, a coating, a foil, a diaper, a wastebag, a tire, a package, a single use article, a protein purification matrix, an implant part, a bone replacement, a slow release drug or a fertilizer or a pesticide carrier comprising use of a biopolymer, renewable polymer or biodegradable polymer or monomer thereof made by a method as provided herein, and optionally the biodegradable, disposable or renewable product is a multidose syringe, a specimen tube, a scalpel, a lancet, a sharps container, or a suction.

In alternative embodiments, PHAs made using products of manufacture, methods and/or bacterial mixtures or consortia as provided herein are used in the production of ethyl 3-ethoxybutyrate (EEB) (EEB can be produced from ethanol and PHA), which can be used as a biofuel additive.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1 schematically illustrates an exemplary methods for selecting, growing and characterizing methanotrophic bacteria for PHA mitigation.

FIG. 2A-B illustrates methanotrophic bacteria growth and PHA accumulation:

FIG. 2A graphically illustrates the growth of methanotrophic cultures in 2 L or 4 L bioreactor cultures;

FIG. 2B schematically illustrates PHA preparations, as further discussed in Example 1, below.

FIG. 3 illustrates images of GC/MS chromatograms of 3-hydroxybutyrate standard (sigma) and PHA preparations extracted from methanotrophic strains AM1, OBBP and R24W, as further discussed in Example 1, below.

FIG. 4A-B illustrates images of 1H-NMR spectra of 3-hydroxybutyrate and PHAs produced by methanotrophic and methylotrophic strains: FIG. 4A, 1H-NMR spectra of 3-hydroxybutyrate, and FIG. 4B, PHAs produced by R24-consortium (B), as further discussed in Example 1, below.

FIG. 5A-C illustrate methanotrophic and methylotrophic strains grown on agar culture plates expressing Gfp (left) or Rfp (right) proteins:

FIG. 5A illustrates strain SM2.2W;

FIG. 5B illustrates strain OBBP; and,

FIG. 5C illustrates strain R24W,

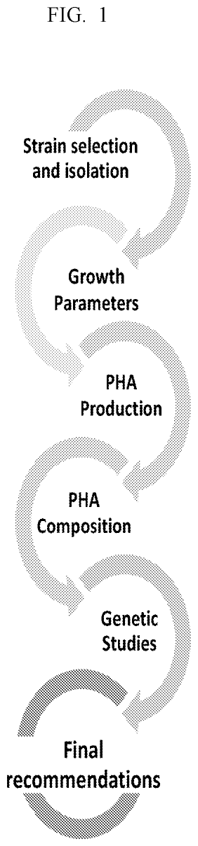

as further discussed in Example 1, below.

Like reference symbols in the various drawings indicate like elements.

### DETAILED DESCRIPTION

In alternative embodiments, provided are mixtures or consortia of polyhydroxyalkanoate (PHA)-producing (optionally polyhydroxybutyrate (PHB)-producing) methanotrophic bacterium, and methods for using them, including methods for making biopolymers, renewable polymers and biodegradable polymers comprising a PHA such as PHB.

In alternative embodiments, products of manufacture as provided herein, including the mixtures or consortia of PHA-producing methanotrophic bacteria as provided herein, are manufactured or configured as arrays, macro- or nano-particles, microfibers, microtubes, microribbons and/or microbeads, where alternatively the macro- or nano-particles, microfibers, microtubes, microribbons and/or microbeads are arranged in arrays. In alternative embodiments, living, active methane-capturing bioagents, including the mixtures or consortia of PHA-producing methanotrophic bacteria as provided herein, optionally comprising halophilic methanotroph cells, are contained in and/or on or are immobilized (directly or indirectly, covalently or non-covalently) in and/or on the arrays, macro- or nano-particles, microfibers, microtubes, microribbons and/or microbeads or nanobeads or equivalents.

In alternative embodiments, living, active methane-capturing bioagents, including the mixtures or consortia of PHA-producing methanotrophic bacteria as provided herein, optionally comprising halophilic methanotroph cells, are contained in and/or on or are immobilized (directly or indirectly, covalently or non-covalently) in and/or on a reaction vessel or a bioreactor.

In alternative embodiments, mixtures or consortia of PHA-producing methanotrophic bacteria as provided herein, including methane-capturing bioagents (methanotrophic bacteria, membranes, or enzymes, including for example, halophilic methanotrophs), are enclosed in or immobilized in or onto a crystal gel matrix, a nanoshell, nanoparticle or equivalent. In alternative embodiments, the mixtures or consortia of PHA-producing methanotrophic bacteria as provided herein, are encapsulated or enclosed or immobilized in and/or on a polymer, a colloidal particle shell, an agar or a gel such as a hydrogel or equivalents. In alternative embodiments, the structures into which the mixtures or consortia of PHA-producing methanotrophic bacteria as provided herein are encapsulated (for example, nanoshells or nanoparticles) are immobilized onto the arrays, macro- or nano-particles, microfibers, microtubes, microribbons and/or microbeads, or equivalents.

In alternative embodiments, the nanoshells are oxide nanoshells such as hollow silica nanoshells, or are metal nanoshells such as gold and silver nanoshells. In alternative embodiments, the nanoparticles comprise CdSe nanoparticles coated with CdS or ZnTe and CdTe nanoparticles coated with CdSe. In alternative embodiments, gold nanoshells (AuNShs) comprise a silica core coated by a thin gold metallic shell.

In alternative embodiments, the microparticles or nanoparticles comprise or are manufactured as plasmonic particles, and the microparticles or nanoparticles can comprise or have an exterior coating comprising: graphene, graphene oxide, reduced graphene oxide, polyethylene glycol (PEG), silica, silica-oxide, polyvinylpyrrolidone, polystyrene, silica, silver, polyvinylpyrrolidone (PVP), cetyl trimethylammonium bromide (CTAB), citrate, lipoic acid, a short chain polyethylenimine (PI), a branched polyethylenimine, reduced graphene oxide, a protein, a peptide, a glycosaminoglycan, or any combination thereof. The nanoparticles, crystal gel matrices or nanoshells can be made by any method, for example, as described in U.S. Pat. No. 9,991,458.

In alternative embodiments, the arrays, macro- or nano-particles, microfibers, microtubes, microribbons and/or microbeads are themselves contained in or fabricated as sheets, mats, meshes, cartridges or any form of secondary or tertiary structure to support the arrays, macro- or nano-particles, microfibers, microtubes, microribbons and/or microbeads, or the immobilized mixtures or consortia of PHA-producing methanotrophic bacteria as provided herein.

In alternative embodiments, the arrays, macro- or nano-particles, microfibers, microtubes, microribbons and/or microbeads, or sheets, meshes, mats, cartridges or any form of secondary structure, are fabricated into modular units or cartridges that can be inserted into a superstructure or device (such as for example a bioreactor), for example, the modular units or cartridges can be fabricated to be exchanged or inserted into a preformed receptacle in or on the superstructure or device, for example to replace an older unit with a newer, fresh unit or cartridge. In alternative embodiments the modular units or cartridges or equivalent structures are fabricated to have gas input and output openings or orifices; for example, gases such as methane-comprising air are fed into the modular unit, cartridge or equivalent structure under pressure such that the air or gas passes through the modular unit, cartridge or equivalent structure and can interact with exemplary LEAFs within the modular unit, cartridge or equivalent structure.

In alternative embodiments, the arrays, macro- or nano-particles, microfibers, microtubes, microribbons and/or microbeads configured as immobilized, active mixtures or consortia of PHA-producing methanotrophic bacteria as provided herein, are a cheap, simple, scalable, cartridge-like system for capturing methane.

In alternative embodiments, mixtures or consortia of PHA-producing methanotrophic bacteria as provided herein, or methane-capturing bioagents such as halophilic methanotroph cells, are used in products of manufacture, and they can comprise bacteria of the genus Methylomicrobium (also known as Methylotuvimicrobium, Methylobacter), for example, can comprise M. buryatenses, M. pelagicum and/or M. alcaliphilum, and optionally the M. alcaliphilum can comprise the species/strain M. alcaliphilum sp. 20Z or M. alcaliphilum 20ZR, and optionally the M. buryatenses can comprise species/strain M. buryatenses 5G.

### Bioreactors

In alternative embodiments, provided are products of manufacture comprising or fabricated as bioreactors for cultivating or culturing non-natural mixtures of consortia of bacterial as provided herein, or for making biopolymers, renewable polymers and biodegradable polymers such as polyhydroxyalkanoate (PHA) such as polyhydroxybutyrate (PHB) and co-polymers.

In alternative embodiments, any product of manufacture, bioreactor, or bioreactor component, can be used to practice products of manufacture or methods as provided herein, for example, as described in: U.S. Pat. Nos. 10,926,261, describing bioreactors based on microfluidic technology; 10,883,074, describing bioreactors having a built in gas distributor, 10,876,087, describing modular tubular bioreactor systems; 10,731,117, describing bioreactors having gas feeding systems; or 10,544,387, 10,400,207, 10,335,751 and 10,280,393, describing making and using bioreactors; and/or, U.S. patent application nos. US20210078005A1, describing macro-sized microbioreactors having an arrangement of microfluidic channels; US 20210024868A1 describing packed-bed bioreactor systems; or, US20210009936A1, describing fluid pumping and bioreactor system including at least two cassettes.

In alternative embodiments, fed-batch (or fed-batch fermentations, as described for example by Nygaard et al, Heliyon, Vol 7(1), January 2021, e05979), continuous culture and/or semi-continuous (cyclic) bioreactors and cultivators, air-lift reactors, continuous stirred tank reactors (CSTR)), and related operation strategies are used. In continuous cultures (as described for example by Blunt et al. Polymers 2018, 10(11), 1197) fresh medium is constantly supplied to the bioreactor, and a portion of the culture is removed at the same rate: the dilution rate. The conditions inside the reactor (substrate, cell, and product concentrations) remain at steady state, and because of this, continuous cultures are often referred to as chemostats, which is short for “chemical environment is static”. Steady-state operation has a number of advantages. It is an attractive platform to study bioprocess physiology and implement control strategies due to the constant growth rate, and is less laborious to operate and maintain once steady-state operation is reached. Steady-state operation also circumvents the major challenge of the optimal control of fed-batch strategies, which involves predicting growth rates over time and under highly dynamic conditions.

Batch and fed-batch processes can be combined to offset low PHA content obtained by each process individually. Under this strategy, the process is divided into two stages: in the first stage the microorganism is grown under batch mode until the desired biomass is achieved and PHA accumulation has started. In the second stage the fermentation is shifted to fed-batch, where usually one or more essential nutrients (most common is nitrogen) are maintained in limited concentration and carbon source is continuously fed into the reactor to further produce and accumulate PHA in the cells.

Continuous culture, or chemostat, is an alternative operation strategy for PHA production. In this method the culture broth is continuously replaced by sterile medium. In chemostat culture, the carbon source is continuously fed in excess, keeping one or more nutrients (e.g. phosphorous or nitrogen) in limitation. Chemostat is highly controllable as the specific growth-rate can be maintained by adjusting the dilution-rate. Therefore, under appropriate growth conditions, continuous fermentation might have the potential to give highest PHA productivity levels.

### Identification of 16 S Ribosomal Nucleic Acid Sequences by Sequence Identity

In alternative embodiments, provided are non-natural mixtures of bacteria and consortia of bacteria identified by having a 16 S ribosomal RNA (rRNA) sequence comprising at least about 90%, 95%, 96%, 97% or 98% or more, or complete (100%), sequence identity to a specific, exemplary 16S rRNA sequence as set forth herein.

In alternative embodiments, “percent (%) nucleic acid or amino acid sequence identity” with respect to a reference nucleic acid or polypeptide sequence is defined as the percentage of nucleic acid residues in a candidate sequence that are identical with nucleic acid or amino acid residues in the nucleic acid sequence, after aligning the sequences and introducing gaps, if necessary, to achieve the maximum percent sequence identity, and not considering any conservative substitutions as part of the sequence identity. Alignment for purposes of determining percent nucleic acid acid sequence identity can be achieved in various ways that are within the skill in the art, for instance, using publicly available computer software such as GAP™ (Genetics Computer Group, University of Wisconsin, Madison, Wis.) (see for example, Devereux et al., Nucl. Acid. Res., 12:387 (1984), BLASTP™, BLASTN™, BLAST™, BLAST-2™, WU-BLAST2/BLAST v2.0 (see for example, Altschul et al. (1996) Methods Enzymol. 266, 460-480), FASTA™ (see for example, Altschul et al., J. Mol. Biol. (1990) 215:403-410), ALIGN™ (Genentech), MEGALIGN™ (DNASTAR) software, or software for executing the Smith-Waterman algorithm or the Needleman-Wunsch algorithm. Those skilled in the art can determine appropriate parameters for aligning sequences, including any algorithms needed to achieve maximal alignment over the full length of the sequences being compared.

In alternative embodiments, align methods comprise use of a BLAST™ analysis employing: (i) a scoring matrix (such as, e.g., BLOSSUM 62™ or PAM 120™) to assign a weighted homology value to each residue and (ii) a filtering program(s) (such as SEG™ or XNU™) that recognizes and eliminates highly repeated sequences from the calculation. In alternative embodiments, align methods comprise use of a BLAST™ analysis employing a BLAST version 2.2.2 algorithm where a filtering setting is set to blastall -p blastp -d “nr pataa”-F F, and all other options are set to default.

In alternative embodiments, alignment schemes for aligning nucleic acid or two amino acid sequences may result in the matching of only a short region of the two sequences, and this small aligned region may have very high sequence identity even though there is no significant relationship between the two full-length sequences. Thus, alternative embodiments, an exemplary alignment method comprises use of the GAP program, which can result in an alignment that spans at least 50 contiguous amino acids of the target polypeptide. For example, using the computer algorithm GAP™, two polypeptides for which the percent sequence identity is to be determined are aligned for optimal matching of their respective amino acids (the “matched span”, as determined by the algorithm). In certain embodiments, a gap opening penalty (which is calculated as 3 times. the average diagonal; the “average diagonal” is the average of the diagonal of the comparison matrix being used; the “diagonal” is the score or number assigned to each perfect nucleic acid or amino acid match by the particular comparison matrix) and a gap extension penalty (which is usually 1/10 times the gap opening penalty), as well as a comparison matrix such as PAM 250™ or BLOSUM 62™ are used in conjunction with the algorithm. In alternative embodiments, a standard comparison matrix (see for example Dayhoff et al., Atlas of Protein Sequence and Structure, 5(3)(1978) for the PAM 250 comparison matrix; Henikoff et al., Proc. Natl. Acad. Sci USA, 89:10915-10919 (1992) for the BLOSUM 62™ comparison matrix) is also used by the algorithm. In alternative embodiments, the parameters for a nucleic acid or polypeptide sequence comparison comprise the following: Algorithm: Needleman et al., J. Mol. Biol., 48:443-453 (1970); Comparison matrix: BLOSUM 62™ from Henikoff et al., supra (1992); Gap Penalty: 12; Gap Length Penalty: 4; Threshold of Similarity: 0. In alternative embodiments, the GAP™ program is used with the above parameters. In certain embodiments, the aforementioned parameters are the default parameters for nucleic acid or polypeptide comparisons (along with no penalty for end gaps) using the GAP™ algorithm.

### Genetic Engineering

In alternative embodiments, provided are genetically engineered bacterium (or plurality of bacteria) for use, for example, in a mixture or consortium as provided herein, and the genetically engineered bacterium or bacteria have contained therein at least one heterologous nucleic acid encoding an enzyme involved in biopolymer, renewable polymer or biodegradable polymer, or polyhydroxyalkanoate (PHA) synthesis.

Routine genetic manipulations. Bacteria used in mixture or consortium as provided herein, or to practice methods as provided herein, can be genetically engineered using any known protocol or procedure.

For example, nucleic acid, or DNA, from a bacterium such as a bacteria from a methanotrophic strain can be isolated by the standard phenol: chloroform method. DNEASY PLANT MINI KIT™ (Qiagen) or ULTRACLEAN® MICROBIAL DNA ISOLATION KIT™ (MoBio, USA) also can be used or applied for a quick extraction for, for example, use in routine nucleic acid sequencing or nucleic acid amplification (for example, polymerase chain reaction (PCR)) analysis or tests.

Genetic tractability assessments and manipulations. Versatile broad-host-range (BHR) and promoter-probe vectors that have been previously developed for use in different groups of methylotrophic and methanotrophic cultures (as described for example, by: Marx, et al Microbiology 147, 2065-2075; Ojala D S, et al Methods Enzymol. 495: 99-118; Puri A W, et al Appl Environ Microbiol. 81(5): 1775-81; Hamilton R, et al (2022) C1-proteins prospect for production of industrial proteins and protein-based materials from methane, In Algal Biorefineries and the Circular Bioeconomy, Ed. Mehariya S, et al, Taylor & Francis/CRC) can be used to make genetically engineered bacteria as provided herein or as used in methods as provided herein.

Vectors can be used as genetic tools for producing or enhancing by genetic engineering PIA-producing methanotrophs and hydrogenotrophs, for example, including use of broad-host-range (BHR) vectors, such as cloning vectors pCM132, pMK10, and pAWP89 (see for example, Marx, et al Microbiology 147, 2065-2075; Ojala D S, et al Methods Enzymol. 495: 99-118; Puri A W, et al Appl Environ Microbiol. 81(5): 1775-81), were tested. Plasmid DNAs can be introduced via conjugation using E. coli, such as E. coli S17-1, as a donor strain or electroporation. The donor strain can be grown on an LB-agar medium supplemented with the appropriate antibiotic for conjugation. The recipient strains grown on P0%- agar medium can be mixed with the donor at donor: recipient ratio of 1:1 and plated on the mating plates (P0% media supplemented with 5% Nutrient Broth). Plates can be incubated at 30° C. under methane: air atmosphere (25:75) for 24-48h. Then cells then can be transferred from a mating medium onto selective plates (P0% media supplemented with an antibiotic, for example, 100 ug/ml kanamycin, 30 ug/ml gentamicin). Kanamycin-resistant clones can be generated for all strains tested, including R24W-M, R24Y-H, and SM2.2W, indicating efficient plasmid transfers. The success of heterologous gene expression can be assessed using fluorescent protein (green fluorescent protein (GFP) and green fluorescent protein (GFP)) as reporters. Green (pMGK10-Ptac-gfp) and red (PAWP89-P89-rfp) fluorescent signals were observed for strains OBBP, LW4, R24W, R24Y, and SM2.2.

Any recombination-based strategy (for example, using two-step allelic exchange suicide vectors exchange coupled with the SacB selection or Cre-lox recombinase), as well as CRISPR/CAS9 strategies, can be applied for metabolic engineering of one or both members of the consortia or bacterial mixture as provided herein; this can significantly reduce or eliminate the technical challenges associated with engineering non-canonical microbial factories.

The following strategies can be applied to improve the production of PHA-copolymers:
1. Construct metabolic traits capable of PHA-copolymer (i.e., poly P3HB-co-4HB) production in co-cultures where one member is capable of 4-hydroxybutyrate (4HB) production and secretion.
2. Enhancing fermentation pathways, specifically propionate production, by enhancing carbon flux into the ethyl malonyl pathway via overexpression of the corresponding enzymes and simultaneous expression of the glyoxylate shunt;
3. Enhancing production of PHA by overexpression of the Type II pha-synthesis (for example, see Chek, M. F., et al, Sci Rep 7, 5312 (2017). https://doi.org/10.1038/s41598-017-05509-4).

Scale-up. In alternative embodiments, provided are methods for making a biopolymer, renewable polymer or biodegradable polymer, wherein optionally the biopolymer, renewable polymer or biodegradable polymer comprises: a polyhydroxyalkanoate (PHA), and optionally the PHA comprises a polyhydroxybutyrate (PHB), comprising culturing a non-natural mixture or consortium of bacteria as set forth herein. The methods comprise “scaled up” processes for making the biopolymers, renewable polymers or biodegradable polymers, and any bioreactor design can be used in constructing products of manufacture as provided herein, or for practicing methods as provided herein, for example, as described in: Strong, P. J., et al. Bioresour Technol 215, 314-323. 10.1016/j.biortech.2016.04.099; and Tikhomirova, T. S., et al. Biotechnol Adv 47: 107709. The process of scale-up and high-density fermentation can be achieved by implementing fermentation technologies previously developed for single-cell protein production, including agitator fermenters (small scale), bubble and bubble-airlift fermenters, loop fermenters, and energy-efficient two-phase jet stream bioreactor, as described for example by Hamilton R, et al, in Algal Biorefineries and the Circular Bioeconomy. Ed. Mehariya S, et al Taylor & Francis/CRC. The process optimization may rely on the successful identification of control parameters that can induce PHA accumulation via nitrogen, sulfur, or phosphate limitations.

Any number of different raw materials can be used as carbon sources for PHA production, for example: waste materials used for PHA production may be grouped into six categories: sugar-based media, starch-based media, cellulosic and hemi-cellulosic media, whey-based media and oil and glycerol-based media. One inexpensive carbon source as an industrial waste material is molasses, which can be derived from either sugarcane or beet.

### Kits

Provided are kits comprising products of manufacture as provided herein, including products of manufacture fully assembled as arrays, sheets, microfibers and/or microbeads or particles comprising immobilized, active mixtures or consortia of PHA-producing methanotrophic bacteria as provided herein, are arranged or fabricated as sheets, mats, cartridges or any form of secondary or tertiary structure to support the arrays, particles, sheets. microfibers and/or microbeads, or immobilized bacteria, optionally comprising PHA-producing or halophilic methanotroph cells, wherein the sheets, mats, cartridges and the like can be further fabricated into tertiary structures, such as modular units that can be easily replaced or exchanged on a superstructure or device.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%. 18%. 17%. 16%. 15%. 14%. 13%. 12% 11%, 10%. 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

### EXAMPLES

### Example 1: Isolating Polyhydroxyalkanoate-Producing Methanotrophs

This Example describes isolating and characterizing strains of novel methanotrophics used to make biopolymers, renewable polymers and biodegradable polymers such as polyhydroxyalkanoates (PHA) such as polyhydroxybutyrate (PHB).

Two strains of novel methanotrophic cultures were isolated. The newly isolated cultures were screened side by side with 14 strains of pure cultures of methanotrophic bacteria. The strains were compared in terms of batch growth kinetics (doubling time T_{d}, growth rate−μ, and biomass yield Y_{g/gCH4}), polyhydroxyalkanoate (or PHA) production and genetic tractability. Both, small scale (50-250 ml) batch cultures and large scale (up to 5 L) bioreactor cultures were carried out. Of the 15 cultures tested, 6 six cultures—Methylocystis parvus OBBP, Methylocystis sp. Rockwell (ATCC 49242), Methylosinus sp. PW1, Methylosinus sp. LW4, Methylocystis sp. SM2.2W and methanotrophic consortia R24-displayed growth rates suitable for industrial applications. Four of them, strains OBBP, Rockwell, SM2.2 and R24W, showed acceptable growth yields (0.5-1 g/g_{CH4}). The strains were grown in large bioreactor cultures (2-5 L) with methane as the carbon source. Strain Methylobacterium extorquence AM1, a model methylotrophic PHB producer, was added for comparative studies. The PHA syntheses were carried out on cell samples collected in late exponential phase (OD=10-15). Biomass was lyophilized. The PHA were extracted and purified using a standard chloroform extraction method. The PHA yields were estimated by weight (g PHA per g cell dry weight, CDW). Three strains, OBBP, SM2.2W and R24W showed attractive PHA production yields (greater than (>) 15% wt/wt PHA content). The PHA accumulation in Strain R24W was greater than (>) 2 fold higher than the other microbial cultures.

The PHB polymers extracted from the strains were subjected to DLS/MALS, GC/MS and ¹³C-NMR studies. Both, GC-MS and ¹³C-NMR spectra showed signatures typical for 3-hydroxybutyrate, suggesting that the culture accumulated poly-3-hydroxybutyrate (P3HB) as the main polymer. The molecular weights of the synthesized polymers are now being evaluated using a light scattering approach. Genetic studies with a range of broad host expressing systems (pMK10, pAWP89, and PCM132) were carried out to identify strains amenable to genetic manipulation. From the initial test, the strains OBBP, LW4, R24W, R24Y and SM2.2 were identified as genetically tractable. Furthermore, broad host range (BHR) vectors based on pAWP78:Ptac-GFP (see for example,Ojala D S, et al. (2011) Methods Enzymol. 495: 99-118; and Puri A W, et al. (2015) Appl Environ Microbiol. 81(5): 1775-81) and pCM132:RFP, were integrated into both partners of a R24W and R24Y co-culture, indicating the potential for simultaneous genetic alterations of the co-culture. Based on the experimental evidence for growth rate and yield, PHB accumulation and genetic tractability, cultures OBBP, R24W and SM2.2 were selected for the next step of development.

Strain selection. 16 strains of methanotrophic bacteria and one strain of methylotrophic bacteria were selected for the initial trial studies, as illustrated in Table 1.

TABLE 1
Summary of microbial cultures tested for growth
on C₁-substrates (methane and/or methanol)

| SPECIES | STRAIN | TAXON ID | GENOME |
|---|---|---|---|
| Methylobacterium | AM1 | 272630 | GCA_000022685.1 |
| extorquens ATCC 55366 | | | |
| Methylosinus | OB3B | 595536 | GCA_000178815.2 |
| trichosporium | | | |
| Methylocystis parvus | OBBP | 1134912 | GCA_000283235.1 |
| Methylosinus sporium | ATCC | 428 | N/A |
| Methylocystis sp. ATCC | Rockwell | 622637 | GCA_000188155.3 |
| 49242 | | | |
| Methylosinus sp. | PW1 | 107636 | GCA_000745215.1 |
| Methylosinus sp | LW3 | 107635 | GCA_000527115.1 |
| Methylosinus sp. | LW4 | 136993 | GCA_000379125.1 |
| Methylocystis sp. | LW5 | 107633 | GCA_000685825.1 |
| Methylosinus sp. | LW7 | 425 | N/A |
| Methylosinus sp. | LW8 | 136994 | N/A |
| Methylosinus sp. | SUV-3W | 2485168 | GCA_004366495.1 |
| Methylosinus sp. | SUV-3B | 2485168 | N/A |
| Methylocaldum sp. | 0917 | 2485163 | GCA_004363855.1 |
| Methylocystis & | R24W* | 133 & 47420 | NA |
| Hydrogenophaga | | | |
| Methylocystis & | R24Y* | 133 & 47420 | NA |
| Hydrogenophaga | | | |
| Methylocystis sp. (90%) | SM2.2W | 133 | NA |

The selected cultures can be divided into four main categories: (1) model cultures, included five strains of microbial cultures, all with demonstrated abilities to utilize C1-substrates (methane or methanol) and produce PHB; (2) Microbial strains which are predicted to produce PHB based on their phylogenic (Type II methanotrophs) and genetic features (the PHB biosynthesis genes); (3) novel microbial isolates belonging to Alphaproteobacterial methanotrophs (Type II) and predicted to produce PHB, and (4) newly isolated microbial cultures.

Enrichment and pure culture isolation studies were carried-out with soil samples obtained from agricultural fields during crop production when the fields were flooded for some period of time. The enrichment culture studies yielded two additional microbial consortia (R24W and R24Y), which were included in the screening.

Except for Methylocaldum sp.0917 (Gammaproteobacterium, Type X methanotrophs), all pure methanotrophic strains selected for this study were Alphaproteobacteria (Type II). Most type II methanotrophs accumulate PHAs a carbon-storage compound (1-2). The methanotrophs can accumulate up to 51% PHA and are often described as promising microbial platforms for production of PHA from methane (3). The methanotrophic consortia, R24W and R24Y, represent stable cocultures of two microbial species, Methylocystis sp (Alphaproteobacterial methanotroph) and Hydrogenophaga (Betaproteobacterial mixotroph). Both, Methylocystic sp R24W/R24Y and Hydrogenophaga sp R24W/R24Y, were only distantly related to known microbial clades, sharing greater than 91% 16S rRNA identity with cultivated pure cultures. Both Methylocystic and Hydrogenophaga are known to produce PHAs.

A Methylobacterium extorquense AM1 culture was also included in the PHB extraction/analysis tests. The strain AM1 has been used as a model system for understanding PHB biosynthesis from single-carbon substrates (4-5). A great deal of information is available for PHB production in the strain from bench to pilot scales (6-8). The strain has been reported to accumulate 40% PHB. The Methylobacterium extorquense AM1 was used mostly for comparative studies.

Enrichment culture. Soil samples (5 g) were inoculated into 125 ml flasks containing 25 ml of nitrate saline media (P0%) and supplemented with 5 ml of methane. The flasks were incubated for 2 days at room temperature with shaking (125 r.p.m. (RPM)). In subsequent enrichments, 10 ml of the previous enrichment culture were diluted 1:10 (to a total of 25 ml) in the same medium and supplemented with 20 ml of methane. Flasks were incubated at room temperature with shaking for 1 week. Cell cultures were transferred at least two more times before plating onto solid media.

The strains R24W and R24Y were obtained as individual colonies (white appearance for RZ24W and yellow colonies for R24Y) after the final enrichment culture was plated onto a solid medium (P0% with 1.2% Bacto agar) and incubated at room temperature for 1 week under methane:air atmosphere (20:80). Each strain was further purified by triplicate streaking on agar plates. While the colonies' appearance differs for two obtained lineages, both cultures consisted of two microbial species, Methylocystic and Hydrogenophaga, based on 16SrRNA sequencing analyses. The strains formed a stable co-culture (consortium).

Strain cultivation. All strains were grown in nitrate mineral media (P0%) containing (g/L): KNO₃, 1, MgSO₄·7H₂O, 0.2, CaCl₂·H₂O, 0.02 trace solution, 1 ml/L and supplemented with 50 ml/L of phosphate solution (5.44 g KH₂PO₄, 5.68 g Na₂HPO₄). The trace elements solution (1 L) contained 0.5 g·Na₂-EDTA, 2.0 g·FeSO₄·7H₂O, 0.3 g·ZnSO₄·7H₂O, 0.03 g·MnCl₂·4H₂O, 0.03 g·H₃BO₃, 0.2 g·CoCl₂·6H₂O, 1.2 g·CuSO₄·5H₂O, 0.5 g·CuCl₂·2H₂O, 0.05 g·NiCl₂·6H₂O, 0.3 g Na₂O₄W×2H₂O and 0.05 g·Na₂MoO₄·2H₂O.

All strains (except Strain AM1, which is non-methanotrophic methylotroph) were initially tested for growth and methane conversion kinetics using small-scale batch cultures (250 ml bottles with 50 ml growth media). The bottles were sealed with rubber stoppers and aluminum caps, then 50 mL of methane was added to the 200 mL headspace. Bottles were shaken at 250 RPM at 30° C. for 1 to 4 days. Cultures were also grown as batch cultures (in triplicate). In all cases the concentrations of CH₄, O₂, and CO₂ in the headspace were measured. The data were analyzed to assess yield (Y), growth rate, and O₂/substrate ratios (Table 2).

TABLE 2
Growth and methane consumption parameters for methanotroph strains *

| | | | GROWTH | O₂/CH₄ | YIELD |
|---|---|---|---|---|---|
| CULTURE | STRAIN | Td (h) | RATE (h⁻¹) | RATIO | (g/g_{CH4}) |
| Methylosinus | OB3B | 11.5 ± 2 | 0.06 ± 0.01 | 1.47 ± 0.03 | 0.9 |
| trichosporium | | | | | |
| Methylocystis parvus | OBBP | 6.7 ± 1 | 0.11 ± 0.01 | 1.45 ± 0.06 | 1.3 |
| Methylosinus sporium | ATCC | 8.5 ± 0.2 | 0.08 ± 0.002 | 1.51 ± 0.05 | 0.7 |
| Methylocystis sp. | Rockwell | 7.2 ± 1 | 0.1 ± 0.01 | 1.39 ± 0.06 | 0.8 |
| Methylosinus sp. | PW1 | 7 ± 0.2 | 0.1 ± 0.003 | 1.46 ± 0.04 | 0.4 |
| Methylosinus sp | LW3 | 12 ± 0.1 | 0.06 ± 0.001 | 1.74 ± 0.27 | 0.6 |
| Methylosinus sp. | LW4 | 6.7 ± 0.4 | 0.1 ± 0.01 | 1.39 ± 0.03 | 0.40 |
| Methylocystis sp. | LW5 | 11.3 ± 0.3 | 0.06 ± 0.01 | 1.57 ± 0.19 | 0.5 |
| Methylosinus sp. | LW7 | 12.3 ± 0.6 | 0.06 ± 0.01 | 1.77 ± 0.13 | 0.7 |
| Methylosinus sp. | LW8 | 17.5 ± 2.3 | 0.03 ± 0.01 | 1.69 ± 0.15 | 0.4 |
| Methylosinus sp. | SUV-3W | 11.2 ± 1 | 0.1 ± 0.01 | 1.55 ± 0.01 | 0.5 |
| Methylosinus sp. | SUV-3B | 11.85 ± 0.6 | 0.1 ± 0.002 | 1.55 ± 0.02 | 0.5 |
| Methylocaldum sp. | O917 | 24.5 ± 5.6 | 0.03 ± 0 | 1.47 ± 0.15 | 0.7 |
| Methylocystis & | R24W | 7.4 ± 0.5 | 0.1 ± 0 | 1.36 ± 0.11 | 0.6 |
| Hydrogenophaga sp | | | | | |
| Methylocystis & | R24Y | 26.8 ± 2.2 | 0.02 ± 0.01 | 1.27 ± 0.31 | 0.4 |
| Hydrogenophaga | | | | | |
| Methylocystis sp. | SS2.2W | 7.1 ± 0.6 | 0.1 ± 0.01 | 1.4 ± 0.24 | 0.7 |

* Strains with attractive growth rate parameters are shown in bold. The strains were selected for further studies in batch bioreactor culture.

The doubling time of the methanotrophic cultures ranged from 6.7 hours (h) to 27 h. The biomass yield also varied significantly, from 0.3 gram (g) to 1 g per g methane consumed. The oxygen: methane consumption data fell in the typical rage, from 1.3 to 1.7. Based on the initial performance in the batch cultures, strains Methylocystis parvus OBBP (Td=6.7, Y=1.3), Methylocystis sp. ATCC 49242 (Td=7.2, Y=0.8), Isolate R24W (Td=7.4, Y=0.6) and Methylocystis sp.SM2.2W (Y=7.1, Y=0.7) were selected for the next PHA synthesis assessment stage.

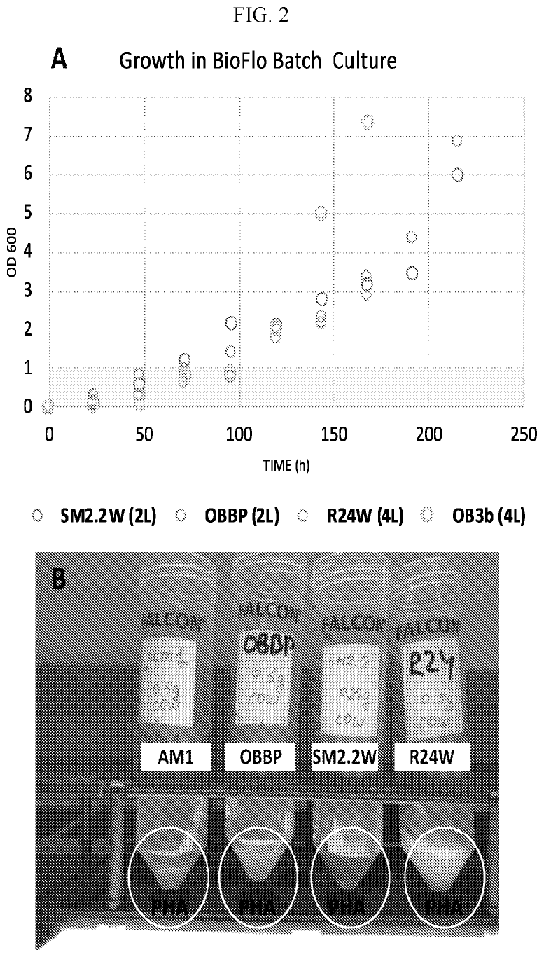

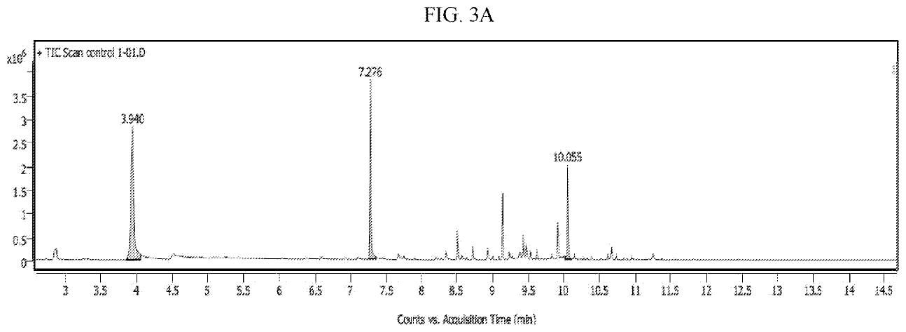

Fermentation in 2-5 L bioreactor cultures. The strains were grown in New Brunswick BIOFLO™ (BioFlo™) Fermenters (2 or 4 L cultures) with methane as the carbon source, using P0% as growth media. The growth curves of the cultures are shown in FIG. 2A.

The summary of bioreactor runs is presented in Table 3. Cells were collected by centrifugation at 4° C. for 20 min at 4700 rpm, washed with phosphate saline buffer (pH 7.4, 137 mM NaCl, 2.7 mM KCl, 10 mM Na₂HPO₄, and 1.8 mM KH₂PO₄) and transferred into 50 ml tubes. Cells were concentrated again using centrifugation and frozen at −20° C. prior to lyophilization. The weight of wet and freeze-dry cells were recorded. At least 0.5 g DCW were obtained for each strain prior to PHB extraction. PHA extraction were carried in triplicates, using an established protocol (1). The purified PHA preparing were weighted again to estimate PHA yields (Table 3). The sufficient PHA materials were obtained for culture AM1, OBBP, SM2.2W and R24W. The co-culture R24W accumulated at least 2.5fold more PHA than the rest (FIG. 2B).

TABLE 3
Summary of bioreactor runs and PHA yield

| | Production | | | PHA (g/g |
|---|---|---|---|---|
| STRAIN | time (days)* | WCW (g/L) | DCW (g/L) | DCW) |
| AM1 | 4 | 1 | 0.25 | 0.15 |
| OBBP | 9 | 3.4 | 1.3 | 0.15 |
| OB3B | 9 | 15.6 | 3 | 0.6 |
| SM2.2 | 10 | 5 | 1 | 0.05 |
| R24W | 8 | 5 | 2 | 0.3 |

* Production time is calculated as time between inoculation and biomass collection

Extracted PHB preparations were subjected for further characterization using GC/MS and ¹³C-NMR to estimate the concentration and molecular composition of the polymers. The molecular size of the polymers can be measured using light scattering protocols.

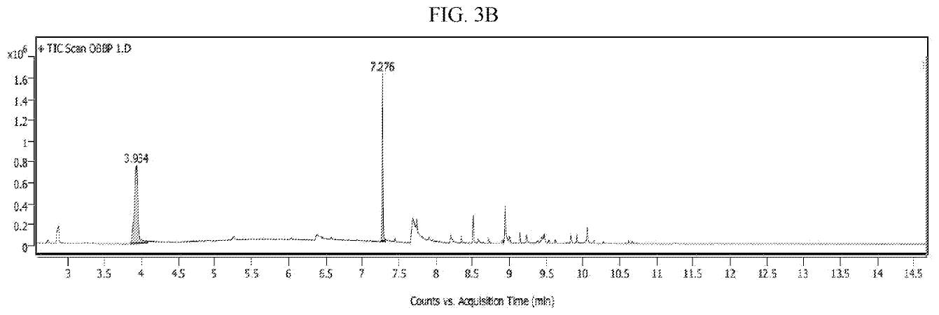

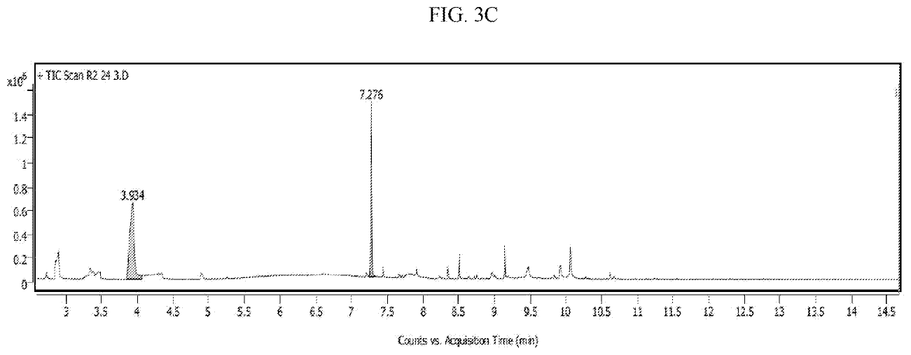

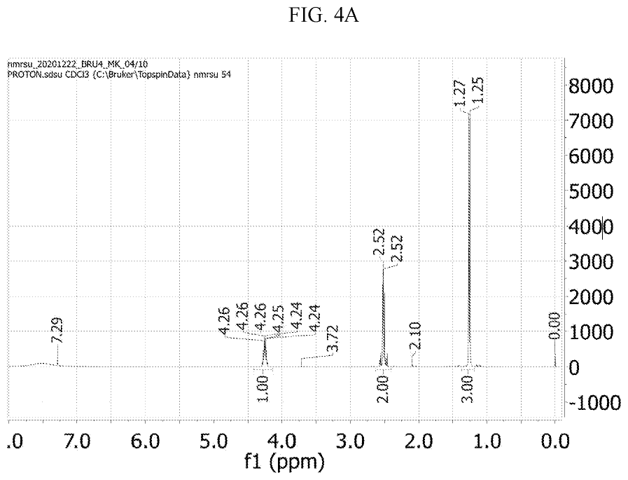

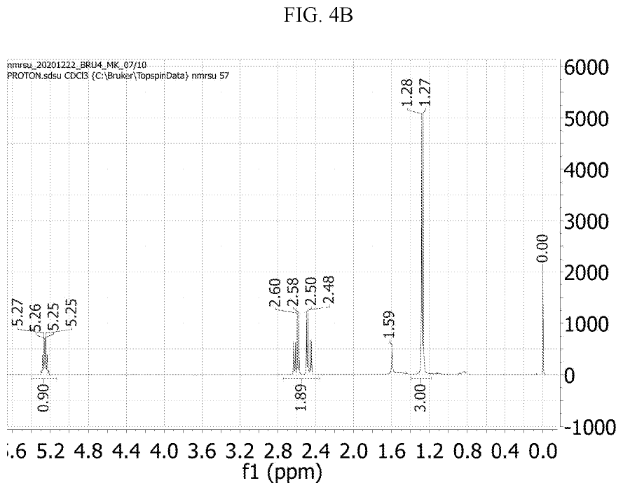

The MS spectra are shown in FIG. 3. The NMR spectra are summarized in FIG. 4. All tested strains accumulate poly-3-hydrohybutyrate as a polymer. No signatures of copolymers were observed.

Metabolic engineering approaches are proven strategies for targeted enhancement of polymer production. However, the success of the metabolic engineering relies on the genetic tractability, or compliance to genetic manipulations, of the selected producer. All strains summarized in Table 1 were tested for gene expression, stability of heterologous genes, and applicability of already available genetic markers and tools. We also evaluated a range of applicable genetic markers (i.e., antibiotic resistance and fluorescent protein expression).

Antibiotic Resistance Tests: To identify genetic markers suitable for genetic manipulation of PHA producing cultures, an antibiotic-resistance screening using antibiotic disks was performed. All strains were plated onto solid P0% media by spreading, then up to 4 antibiotic disks were placed on top. The plates were incubated under methane:air for one week and then were evaluated. The outcomes of antibiotic-resistance tests are presented in Table 4.

TABLE 4
Antibiotic susceptibility testing (AST) results

| | Streptomycin | Pencillin | Gentamicin | Chlorophenicol | Tetracyclin | Neomycin | Erythrocyclin | Ampicillin | Kanamycin |
|---|---|---|---|---|---|---|---|---|---|
| Strain | (10 mcg) | (10 U) | (10 mcg) | (30 mcg) | (30 mcg) | (30 mcg) | (15 mcg) | (10 mcg) | (25 mcg) |
| | | | | | | | | | X |
| OB3B | | | | | | | | | X |
| ATCC | | | | | | | | | X |
| ROCKWELL | | | | | | | | | X |
| PW1 | X | X | X | X | X | X | X | R | X |
| LW3 | | R | | | | | | R | X |
| LW4 | X | R | X | R | R | X | R | R | X |
| LW5 | X | R | R | | R | X | R | X | X |
| LW7 | X | X | X | X | R | | X | X | X |
| LW8 | Resistant | | | | X | | | R | X |
| SUV#3B | | | | | | | R | | X |
| SUV#3W | X | R | X | X | R | X | X | | X |
| R24Y | X | R | X | R | X | R | X | | X |
| R24W | | | | | | | | | X |
| SM2.2W | X | | X | X | R | R | X | | X |

Routine genetic manipulations. DNA from methanotrophic strains could be isolated by a standard phenol:chloroform method. DNEASY PLANT MINI KIT® (Qiagen) or ULTRACLEAN® MICROBIAL DNA ISOLATION KIT® (MoBio, USA) were also applied for a quick extraction for routine PCR tests.

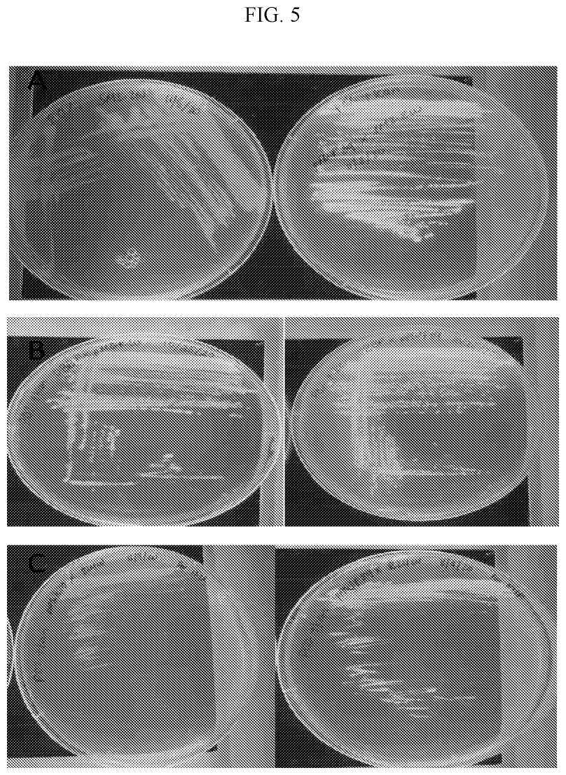

Genetic tractability assessments. A variety of versatile broad-host-range (BHR) and promoter-probe vectors have been previously developed for use in different groups of methylotrophic and methanotrophic cultures (10-12). To validate the applicability of the vectors as genetic tools for the PHA-producing methanotrophs, previously developed HHR vectors, such as cloning vectors pCM132, pMK10 and pAWP89 (10-12), were tested. Plasmid DNAs were introduced into methanotroph hosts via conjugation using E. coli S17-1 as a donor strain. The donor strain was grown on LB-agar medium supplemented with the appropriate antibiotic and the recipient strains rown on P0%- agar medium were mixed in a donor: recipient ratio of 1:1 and plated on the mating plates (P0% media supplemented with 5% Nutrient Broth). Plates were incubated at 30° C. under a methane:air atmosphere (25:75) for 48h, and then cells were transferred from a mating medium onto selective plates (P0% media supplemented with 100 ug/ml kanamycin). Kanamycin-resistant clones were generated for strains OBBP, LW4, R24W, R24Y, and SM2.2W indicating efficient plasmid transfers. The success of heterologous gene expression was assessed using fluorescent protein (including green fluorescent protein (GFP) and red fluorescent protein (RFP)) as reporters. Green (pMGK10-P_{tac}-gfp) and red (PAWP89-P₈₉-rfp) fluorescent signals were observed for strains OBBP, LW4, R24W, R24Y, and SM2.2 (FIG. 5).

### REFERENCES

1. Wendlandt, K. D., et al., U. Production of PHB with a high molecular mass from methane. Polym. Degrad. Stabil. 1998, 59, 191-194.
2. Lu-Yao Liu, et al., Biological conversion of methane to polyhydroxyalkanoates: Current advances, challenges, and perspectives, Environmental Science and Ecotechnology, 2, 2020, 100029, ISSN 2666-4984, https://doi.org/10.1016/j.ese.2020.100029.
3. Tan, Giin-Yu, et al. (2014). Start a Research on Biopolymer Polyhydroxyalkanoate (PHA): A Review. Polymers. 6. 706-754. 10.3390/polym6030706.
4. Povolo S, et al., Polyhydroxyalkanoate biosynthesis by Hydrogenophaga pseudoflava DSM1034 from structurally unrelated carbon sources. N Biotechnol. 2013. 30(6):629-34. doi: 10.1016/j.nbt.2012.11.019. Epub 2012 November 29. PMID: 23201074.
5. Korotkova N, et al., Poly-beta-hydroxybutyrate biosynthesis in the facultative methylotroph Methylobacterium extorquens AM1: identification and mutation of gapl1, gap20, and phaR. J Bacteriol. 2002 184(22):6174-81. doi:
6. Ochsner A M, et al., Methylobacterium extorquens: methylotrophy and biotechnological applications. Appl Microbiol Biotechnol. 2015 January; 99(2):517-34. doi: 10.1007/s00253-014-6240-3. Epub 2014 November 30. PMID: 25432674.
7. Bourque, D., et al., High-cell-density production of poly-β-hydroxybutyrate (PHB) from methanol by Methylobacterium extorquens: Production of high-molecular-mass PHB. Appl. Microbiol. Biotechnol. 1995, 44, 367-376.
8. Mokhtari-Hosseini, Z. B., et al., Statistical media optimization for growth and PHB production from methanol by a methylotrophic bacterium. Bioresour. Technol. 2009, 100, 2436-2443.
9. Mokhtari-Hosseini, Z. B., et al., A. Effect of feed composition on PHB production from methanol by HCDC of Methylobacterium extorquens (DSMZ 1340). J. Chem. Technol. Biotechnol. 2009, 84, 1136-1139.
10. Marx, C. J. et al. (2001). Development of improved versatile broad-host-range vectors for use in methylotrophs and other Gram-negative bacteria. Microbiology 147, 2065-2075.
11. Ojala D S, et al. 2011. Genetic systems for moderately halo(alkali)philic bacteria of the genus Methylomicrobium. Methods Enzymol. 495: 99-118.
12. Puri A W, et al., 2015. Genetic tools for the industrially promising methanotroph Methylomicrobium buryatense. Appl Environ Microbiol. 81(5): 1775-81.

A number of embodiments of the invention have been described. Nevertheless, it can be understood that various modifications may be made without departing from the spirit and scope of the invention. Accordingly, other embodiments are within the scope of the following claims.

1-38. (canceled)

39. A non-natural mixture or consortium of bacteria, comprising:
a polyhydroxyalkanoate (PHA)-producing methanotrophic bacterium or biopolymer-, renewable polymer- or biodegradable polymer-producing methanotrophic bacterium; and
a polyhydroxyalkanoate (PHA)-producing aerobic chemolithoautotrophy bacterium or a biopolymer-, renewable polymer- or biodegradable polymer-producing aerobic chemolithoautotrophy bacterium.

40. The non-natural mixture or consortium of bacteria of claim 39, wherein the PHA comprises polyhydroxybutyrate (PHB), or the biopolymer, renewable polymer or biodegradable polymer comprises a polyhydroxyalkanoate (PHA), a poly(3-hydroxypropionate) (PHP or P3HP), a poly(3-hydroxybutyrate) (PHB or P3HB), a poly(4-hydroxybutyrate) (P4HB), a poly(3-hydroxyvalerate) (PHV or P3HV), a poly(4-hydroxyvalerate) (P4HV), a poly(5-hydroxyvalerate) (P5HV), a poly(3-hydroxyhexanoate) (PHHx or P3HHx), a poly(3-hydroxyoctanoate) (PHO, or P3HO), a poly(3-hydroxydecanoate) (PHD or P3HD), a poly(3-hydroxyundecanoate) (PHU, P3HU), a short- or medium-chain length, saturated or unsaturated PHA, a polylactic acid (PLA), any copolymer thereof, or any combination thereof.

41. The non-natural mixture or consortium of bacteria of claim 39, wherein the polyhydroxyalkanoate (PHA)-producing methanotrophic bacterium or biopolymer-, renewable polymer- or biodegradable polymer-producing methanotrophic bacterium are of or classified in (or derived from) the genus Methylobacterium, Methylosinus, Methylocella, Methylocapsa, Methylorubrum or Methylocystis.

42. The non-natural mixture or consortium of bacteria of claim 39, wherein the polyhydroxyalkanoate (PHA)-producing aerobic chemolithoautotrophy bacterium or the biopolymer-, renewable polymer- or biodegradable polymer-producing aerobic chemolithoautotrophy bacterium are of or classified in (or derived from) the genus Methyloversatilis, Rubrivivax, Rhodopseudomonas, Xanthobacter, Ralstonia, Cuprividus or Hydrogenophaga.

43. The non-natural mixture or consortium of bacteria of claim 39, wherein the non-natural mixture or consortium of bacteria comprises:
at least one bacterium from the genus Methylobacterium and at least one bacterium from the genus Methyloversatilis;
at least one bacterium from the genus Methylobacterium and at least one bacterium from the genus Rubrivivax;
at least one bacterium from the genus Methylobacterium and at least one bacterium from the genus Rhodopseudomonas;
at least one bacterium from the genus Methylobacterium and at least one bacterium from the genus Xanthobacter;
at least one bacterium from the genus Methylobacterium and at least one bacterium from the genus Ralstonia;
at least one bacterium from the genus Methylobacterium and at least one bacterium from the genus Cuprividus; or
at least one bacterium from the genus Methylobacterium and at least one bacterium from the genus Hydrogenophaga;
at least one bacterium from the genus Methylosinus and at least one bacterium from the genus Methyloversatilis;
at least one bacterium from the genus Methylosinus and at least one bacterium from the genus Rubrivivax;
at least one bacterium from the genus Methylosinus and at least one bacterium from the genus Rhodopseudomonas;
at least one bacterium from the genus Methylosinus and at least one bacterium from the genus Xanthobacter;
at least one bacterium from the genus Methylosinus and at least one bacterium from the genus Ralstonia;
at least one bacterium from the genus Methylosinus and at least one bacterium from the genus Cuprividus; or
at least one bacterium from the genus Methylosinus and at least one bacterium from the genus Hydrogenophaga;
at least one bacterium from the genus Methylocella and at least one bacterium from the genus Methyloversatilis;
at least one bacterium from the genus Methylocella and at least one bacterium from the genus Rubrivivax;
at least one bacterium from the genus Methylocella and at least one bacterium from the genus Rhodopseudomonas;
at least one bacterium from the genus Methylocella and at least one bacterium from the genus Xanthobacter;
at least one bacterium from the genus Methylocella and at least one bacterium from the genus Ralstonia;
at least one bacterium from the genus Methylocella and at least one bacterium from the genus Cuprividus; or
at least one bacterium from the genus Methylocella and at least one bacterium from the genus Hydrogenophaga;
at least one bacterium from the genus Methylocella and at least one bacterium from the genus Methyloversatilis;
at least one bacterium from the genus Methylocapsa and at least one bacterium from the genus Rubrivivax;
at least one bacterium from the genus Methylocapsa and at least one bacterium from the genus Rhodopseudomonas;
at least one bacterium from the genus Methylocapsa and at least one bacterium from the genus Xanthobacter;
at least one bacterium from the genus Methylocapsa and at least one bacterium from the genus Ralstonia;
at least one bacterium from the genus Methylocapsa and at least one bacterium from the genus Cuprividus; or
at least one bacterium from the genus Methylocapsa and at least one bacterium from the genus Hydrogenophaga;
at least one bacterium from the genus Methylorubrum and at least one bacterium from the genus Methyloversatilis;
at least one bacterium from the genus Methylorubrum and at least one bacterium from the genus Rubrivivax;
at least one bacterium from the genus Methylorubrum and at least one bacterium from the genus Rhodopseudomonas;
at least one bacterium from the genus Methylorubrum and at least one bacterium from the genus Xanthobacter;
at least one bacterium from the genus Methylorubrum and at least one bacterium from the genus Ralstonia;
at least one bacterium from the genus Methylorubrum and at least one bacterium from the genus Cuprividus;
at least one bacterium from the genus Methylorubrum and at least one bacterium from the genus Hydrogenophaga;
at least one bacterium from the genus Methylocystis and at least one bacterium from the genus Methyloversatilis;
at least one bacterium from the genus Methylocystis and at least one bacterium from the genus Rubrivivax;
at least one bacterium from the genus Methylocystis and at least one bacterium from the genus Rhodopseudomonas;
at least one bacterium from the genus Methylocystis and at least one bacterium from the genus Xanthobacter;
at least one bacterium from the genus Methylocystis and at least one bacterium from the genus Ralstonia;
at least one bacterium from the genus Methylocystis and at least one bacterium from the genus Cuprividus; or
at least one bacterium from the genus Methylocystis and at least one bacterium from the genus Hydrogenophaga.

44. The non-natural mixture or consortium of bacteria of claim 39, wherein the non-natural mixture or consortium of bacteria comprises a contribution of PHA production of at least 80% (PHA biorenewable etc.) by a member from the genus Methylobacterium and a contribution of PHA production of at least 20% by a member from the genus Methyloversatilis.

45. A genetically engineered bacterium, wherein the bacterium is or is derived from a bacterium in a mixture or consortium as set forth in claim 40, where the characteristics of microorganisms are obtained due to mutation, genetically-engineered mutation, and/or selection-pressure-induced mutation having contained therein at least one heterologous nucleic acid encoding an enzyme involved in biopolymer, renewable polymer or biodegradable polymer, or polyhydroxyalkanoate (PHA) synthesis.

46. The genetically engineered bacterium of claim 45, wherein the at least one heterologous nucleic acid encodes a β-ketothiolase or an acetoacetyl-CoA reductase.

47. The genetically engineered bacterium of claim 45, having contained therein a heterologous operon for polyhydroxyalkanoate (PHA) biosynthesis.

48. The non-natural mixture or consortium of bacteria of claim 39, wherein:
(a) at least one polyhydroxyalkanoate (PHA)-producing methanotrophic bacterium or biopolymer-, renewable polymer- or biodegradable polymer-producing methanotrophic bacterium has contained therein at least one heterologous nucleic acid encoding an enzyme involved in biopolymer, renewable polymer or biodegradable polymer or polyhydroxyalkanoate (PHA) synthesis; or
(b) at least one polyhydroxyalkanoate (PHA)-producing aerobic chemolithoautotrophy bacterium or a biopolymer-, renewable polymer- or biodegradable polymer-producing aerobic chemolithoautotrophy bacterium has contained therein at least one heterologous nucleic acid encoding an enzyme involved in biopolymer, renewable polymer or biodegradable polymer or polyhydroxyalkanoate (PHA) synthesis.

49. A product of manufacture comprising the non-natural mixture or consortium of bacteria of claim 39.

50. The product of manufacture of claim 49, wherein the bacterium or the non-natural mixture or consortium of bacteria are attached to or contained in a plurality of macro- or nano-particles, microfibers, microtubes, microribbons and/or microbeads, or are enclosed in or immobilized in or onto a crystal gel matrix or a nanoshell.

51. A method for making a biopolymer, renewable polymer or biodegradable polymer, comprising:
(a) culturing a non-natural mixture or consortium of bacteria comprising a polyhydroxyalkanoate (PHA)-producing methanotrophic bacterium or biopolymer-, renewable polymer- or biodegradable polymer-producing methanotrophic bacterium and comprising a polyhydroxyalkanoate (PHA)-producing aerobic chemolithoautotrophy bacterium or a biopolymer-, renewable polymer- or biodegradable polymer-producing aerobic chemolithoautotrophy bacterium; and
(b) isolating, separating, purifying or harvesting the biopolymer, renewable polymer or biodegradable polymer.

52. The method of claim 51, wherein the bacterium or a mixture or consortium of bacteria are cultured under conditions comprising adding to the culture:
methane and air, methane and oxygen, methane, hydrogen and air, methane and hydrogen, air, or combinations thereof, and
reagents comprising methanol, acetate, formate and/or succinate.

53. The method of claim 51, wherein the biopolymer, renewable polymer or biodegradable polymer is separated, isolated or purified to between about 70% to about 99.5% purity.

54. The method of claim 51, wherein the biopolymer, renewable polymer or biodegradable polymer comprises a poly-4-hydroxybutyrate (P4HB) or a copolymer thereof.

55. A process for producing a monomer from a biopolymer, renewable polymer or biodegradable polymer, comprising:
(a) providing a biopolymer, renewable polymer or biodegradable polymer produced, isolated or separated using the method of claim 51;
(b) providing a depolymerization substance or reagent comprising a thermostable extracellular PHA depolymerase; and
(c) depolymerizing the biopolymer, renewable polymer or biodegradable polymer by admixing the biopolymer, renewable polymer or biodegradable polymer and the depolymerization substance.

56. A biocompatible product, a bioactive nanobead, a biodegradable, disposable or renewable product, a thermoplast, an elastomer product or a polymer precursor, a coating, a foil, a diaper, a wastebag, a tire, a package, a single use article, a protein purification matrix, an implant part, a bone replacement, a slow release drug or a fertilizer or a pesticide carrier comprising using a biopolymer, renewable polymer or biodegradable polymer or monomer thereof made by the method of claim 55.


SEQUENCE LISTING (9 sequences)

SEQ ID NO: 1 - DNA, 1408 bp, Methylocystis
aacgaacgctggcggcaggcctaacacatgcaagtcgaacgctgtagcaatacagagtggcagacgggtgagtaacgcgtgggaacgtgcctttcggttcggaataactcagggaaacttgagctaataccggatacgccctttgggggaaagatttattgccgaaagatcggcccgcgtccgattagctagttggtgtggtaatggcgcaccaaggcgacgatcggtagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcgccagggacgataatgacggtacctggataagaagccccggctaacttcgtgccagcagccgcggtaatacgaagggggctagcgttgttcggaatcactgggcgtaaagcgcacgtaggcggatctttaagtcaggggtgaaatcccgaggctcaacctcggaactgcctttgatactggaggtctcgagtccgggagaggtgagtggaactgcgagtgtagaggtgaaattcgtagatattcgcaagaacaccagtggcgaaggcggctcactggcccggtactgacgctgaggtgcgaaagcgtggggagcaaacaggattagataccctggtagtccacgccgtaaacgatggatgctagccgttggggagcatgctcttcagtggcgcagctaacgctttaagcatcccgcctggggagtacggtcgcaagattaaaactcaaaggaattgacgggggcccgcacaagcggtggagcatgtggtttaattcgaagcaacgcgcagaaccttaccagcttttgacatgcccggtatgatcgccagagatggctttcttcccgcaaggggccggagcacaggtgctgcatggctgtcgtcagctcgtgtcgtgagatgttgggttaagtcccgcaacgagcgcaaccctcgcccttagttgccatcattcagttgggcactctagggggactgccggtgataagccgcgaggaaggtggggatgacgtcaagtcctcatggcccttacaggctgggctacacacgtgctacaatggcggtgacaatgggaagcgaaagggcgacctggagcaaatctcaaaaagccgtctcagttcggattgcactctgcaactcgagtgcatgaaggtggaatcgctagtaatcgcagatcagcacgctgcggtgaatacgttcccgggccttgtacacaccgcccgtcacaccatgggagttggttttacccgaaggcgtttcgccaaccgcaaggaggcaggcgaccacggtagggtcagcgactggggtg

SEQ ID NO: 2 - DNA, 1410 bp, Methylosinus
aacgaacgctggcggcaggcttaacacatgcaagtcgaacgggcgcagcgatgcgtcagtggcagacgggtgagtaacgcgtgggaacgtacctttcggttcggaataactcagggaaacttgagctaataccggatacgcccttagggggaaagatttattgccgaaagatcggcccgcgtccgattagctagttggtgaggtaaaggctcaccaaggcgacgatcggtagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcgccagggacgataatgacggtacctggataagaagccccggctaacttcgtgccagcagccgcggtaatacgaagggggctagcgttgttcggaatcactgggcgtaaagcgcacgtaggcggattgttaagtcaggggtgaaatcccgaggctcaacctcggaactgcctttgatactggcgatctagagtccgggagaggtgagtggaactgcgagtgtagaggtgaaattcgtagatattcgcaagaacaccagtggcgaaggcggctcactggcccggaactgacgctgaggtgcgaaagcgtggggagcaaacaggattagataccctggtagtccacgccgtaaacgatggatgctagccgttggggagcttgctcttcagtggcgcagctaacgctttaagcatcccgcctggggagtacggtcgcaagattaaaactcaaaggaattgacgggggcccgcacaagcggtggagcatgtggtttaattcgaagcaacgcgcagaaccttaccagcttttgacatgtccggtatggtcgtcagagatgacttccttcccgcaaggggccggaacacaggtgctgcatggctgtcgtcagctcgtgtcgtgagatgttgggttaagtcccgcaacgagcgcaaccctcgcccttagttgccatcattcagttgggcactctagggggactgccggtgataagccgcgaggaaggtggggatgacgtcaagtcctcatggcccttacaggctgggctacacacgtgctacaatggcggtgacaatgggaagcgaaggggtgacccggagcaaatctccaaaagccgtctcagttcggattgcactctgcaactcgagtgcatgaaggtggaatcgctagtaatcgcagatcagcacgctgcggtgaatacgttcccgggccttgtacacaccgcccgtcacaccatgggagttggctttacccgaaggcgtttcgctaaccgcaaggaggcagacgaccacggtagggtcagcgactggggtg

SEQ ID NO: 3 - DNA, 1544 bp, Methylocaldum
ttaaactgaagagtttgatcatggctcagattgaacgctggcggcatgcttaacacatgcaagtcgaacggcagcacagccgggtaaccggtgggtggcgagtggcggacgggtgagtaatgcgtaggaatctgccttgtagtgggggataactcggggaaactcgggctaataccgcatacgctctacggaggaaagcgggggatcttcggacctcgcgctatgagatgagcctacgtccgattagctagttggcagggtaatggcctaccaaggcgacgatcggtagctggtctgagaggacgatcagccacactggaactgagacacggtccagactcctacgggaggcagcagtggggaatattggacaatgggcgcaagcctgatccagcaatgccgcgtgtgtgaagaaggcctgcgggttgtaaagcactttaagcagggaagaaaggtggggtgttaacaccatctcacattgacgttacctgcagaataagcaccggctaactccgtgccagcagccgcggtaatacggagggtgcgagcgttaatcggaattactgggcgtaaagcgcgcgtaggcggtttgttaagtcagctgtgaaagccccgggcttaacctgggaatggcagttgatactggcgagctagagtgtggtagaggggtgtggaatttccggtgtagcagtgaaatgcgtagagatcggaaggaacaccagtggcgaaggcggcaccctggaccaacactgacgctgaggtgcgaaagcgtggggagcaaacaggattagataccctggtagtccacgctgtaaacgatgagaactagccgttgggcacaatcgagtgtttagtggcgcagctaacgcgataagttctccgcctggggagtacggccgcaaggttaaaactcaaatgaattgacgggggcccgcacaagcggtggagcatgtggtttaattcgatgcaacgcgaagaaccttacctggtcttgacatgtcgcgaacccttgagagatcgaggggtgccttcgggagcgcgaacacaggtgctgcatggctgtcgtcagctcgtgtcgtgagatgttgggttaagtcccgtaacgagcgcaacccttgtccctagttgccagcgttgagtcgggaactctagggagactgccggtgataaaccggaggaaggtggggatgacgtcaagtcatcatggcccttatgaccagggctacacacgtgctacaatggtcggtacagagggtagccaagccgtgaggcggagccaatctcacaaagccgatcgtagtccggattgcagtctgcaactcgactgcatgaagtcggaatcgctagtaatcgcggatcagcatgccgcggtgaatacgttcccgggccttgtacacaccgcccgtcacaccatgggagtgggttgcaccagaagcaggtagtctaaccttcgggagggcgcttgccacggtgtggttcatgactggggtgaagtcgtaacaaggtagccgtaggggaacctgcggctggatcacctcctttca

SEQ ID NO: 4 - DNA, 1519 bp, Hydrogenophaga
  misc_feature (109)..(109): n is a, c, g, or t
  misc_feature (624)..(624): n is a, c, g, or t
  misc_feature (1133)..(1133): n is a, c, g, or t
agtttgatcctggctcagattgaacgctggcggcatgctttacacatgcaagtcgaacggtaacaggccgcaaggtgctgacgagtggcgaacgggtgagtaatgcatnggaacgtgcccagtcgtgggggataacgcagcgaaagctgtgctaataccgcatacgatctatggatgaaagcgggggaccgtaaggcctcgcgcgattggagcggccgatgtcagattagatagttggtggggtaaaggctcaccaagccaacgatctgtagctggtctgagaggacggccagccacactgggactgagacacggcccagactcctacgggaggcagcagtggggaattttggacaatgggcgcaagcctgatccagcaatgccgcgtgcaggaagaaggccttcgggttgtaaactgcttttgtacggaacgaacggtcttgggttaatacctcgggctaatgacggtaccgtaagaataagcaccggctaactacgtgccagcagccgcggtaatacgtagggtgcaagcgttaatcggaattactgggcgtaaagcgtgcgcaggcggttttgtaagacaggcgtgaaatccccgggctcaacctgggaattgcgcttgtnactgcaaggctggagtgcggcagagggggatggaattccgcgtgtagcagtgaaatgcgtagatatgcggaggaacaccgatggcgaaggcaatcccctgggcctgcactgacgctcatgcacgaaagcgtggggagcaaacaggattagataccctggtagtccacgccctaaacgatgtcaactggttgttgggaatttaccttctcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatggcaggaagtttccagagatggattcgtgctcgaaagagaacctgcacacaggtgctgcatggctgtcgtcagctcgtgtcgtgagatgttgggttaagtcccgcaacgagcgcaacccttgccattagttgctacgaaagggcactctaatgngactgccggtgacaaaccggaggaaggtggggatgacgtcaagtcctcatggcccttataggtggggctacacacgtcatacaatggccggtacaaagggcagccaacccgcgagggggagccaatcccataaagccggtcgtagtccggttcgcagtctgcaactcgactgcgtgaagtcggaatcgctagtaatcgtggatcagcatgtcacggtgaatacgttcccgggtcttgtacacaccgcccgtcacaccatgggagcgggtctcgccagaagtagttagcctaaccgcaaggagggcgattaccacggcggggttcgtgactggggtgaagtcgtaacaaggtagccgtatcggaaggtgcggctggatcacctcctt

SEQ ID NO: 5 - DNA, 1407 bp, Xanthobacter
agcgaacgctggcggcaggcctaacacatgcaagtcgagcgcccagcaatgggagcggcagacgggtgagtaacacgtggggatctacccaatggtacggaataacccagggaaacttggactaataccgtatgtgcccttcgggggaaagatttatcgccattggatgaacccgcgtcggattagctagttggtgaggtaaaggctcaccaaggcgacgatccgtagctggtctgagaggatgatcagccacactgggactgagacacggcccagactcctacgggaggcagcagtggggaatattggacaatgggcgcaagcctgatccagccatgccgcgtgtgtgatgaaggccttagggttgtaaagcactttcgccggtgaagataatgacggtaaccggagaagaagccccggctaacttcgtgccagcagccgcggtaatacgaagggggcaagcgttgctcggaatcactgggcgtaaagcgcacgtaggcgggttgttaagtcagaggtgaaatcctggagctcaactccagaactgcctttgatactggcgaccttgagttcgagagaggttggtggaactgcgagtgtagaggtgaaattcgtagatattcgcaagaacaccagtggcgaaggcggccaactggctcgatactgacgctgaggtgcgaaagcgtggggagcaaacaggattagataccctggtagtccacgccgtaaacgatggatgctagccgttaggcagcttgctgcttagtggcgcagctaacgcattaagcatcccgcctggggagtacggtcgcaagattaaaactcaaaggaattgacgggggcccgcacaagcggtggagcatgtggtttaattcgaagcaacgcgcagaaccttaccagcctttgacatggcaggacgatttccagagatggatctcttccagcaatggacctgcacacaggtgctgcatggctgtcgtcagctcgtgtcgtgagatgttgggttaagtcccgcaacgagcgcaaccctcgcctctagttgccagcattcagttgggcactctagagggactgccggtgataagccgcgaggaaggtggggatgacgtcaagtcctcatggcccttacgggctgggctacacacgtgctacaatggcggtgacagtgggatgcaaaggggcgacccctagcaaatctccaaaagccgtctcagttcggattgcactctgcaactcgagtgcatgaagttggaatcgctagtaatcgtggatcagcatgccacggtgaatacgttcccgggccttgtacacaccgcccgtcacaccatgggagttggctttacccgaaggcgctgcgctaacccgcaagggaggcaggcgaccacggtagggtcagcgactggggtg

SEQ ID NO: 6 - DNA, 1490 bp, Ralstonia
agagtttgatcctggctcagattgaacgctggcggcatgccttacacatgcaagtcgaacggcagcacgggcttcggcctggtggcgagtggcgaacgggtgagtaatacatcggaacgtgccctgtagtgggggataactagtcgaaagattagctaataccgcatacgacctgagggtgaaagcgggggaccgaaggcctcgcgctacaggagcggccgatgtctgattagctagttggtggggtaaagcctaccaaggcgacgatcagtagctggtctgagaggacgatcagccacactgggactgagaacacggcccagactcctacggaggcagcagtggggaattttggacaatgggggcaaccctgatccagcaatgccgcgtgtgtgaagaaggccttcgggtttgtaaagcacttttgtccggaaagaaatggctctggttaatacccggggtcgatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgagctcaacttgggaatggcgcttgtgactgcaaggctagagtatgtcagaggggggtagaattccacgtgtagcagtgaaatgcgtagagatgtggaggaataccgatggcgaaggcagccccctgggacgtcactgacgctcatgcacgaaagcgtggggagcaaacaggattagataccctggtagtccacgccctaaacgatgtcaactagttgttggggattcatttcttcagtaacgtagctaacgcgtgaagttgaccgcctggggagtacggtcgcaagattaaaactcaaaggaattgacggggaccgcacaagcggtggatgatgtggattaattcgatgcaacgcgaaaaaccttacctacccttgacatgccactaacgaagcagagatgcattaggtgtccgaaagggagagtggacacaggtgctgcatggctgtcgtcagctcgtgtcgtgagatgttgggttaagtcccgcaacgagcgcaacccttgtctctagttgctacgaaagggcactctagagagactgccggtgacaaaccggaggaaggtggggatgacgtcaagtcctcatggccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccgggtcttgtacacaccgcccgtcacaccatgggagtgggttttgccagaagtagttagcctaaccgcaaggagggcgattaccacagcagggttcatgactggggtgaagtcgtaacaaggtaacc

SEQ ID NO: 7 - DNA, 1471 bp, Rubrivivax
tcagattgaacgctggcggcatgccttacacatgcaagtcgaacggtaacaggccgcaaggtgctgacgagtggcgaacgggtgagtaatgcatcggaacgtgcccagtagtgggggatagcccggcgaaagccggattaataccgcatacgacctatgggtgaaagcgggggaccgaaaggcctcgcgctattggagcggccgatgtcagattaggtagttggtggggtaaaggcctaccaagcctacgatctgtagctggtctgagaggacgaccagccacactgggactgagacacggcccagactcctacgggaggcagcagtggggaattttggacaatgggcgcaagcctgatccagccatgccgcgtgcgggaagaaggccttcgggttgtaaaccgcttttgtcagggaagaaatcttctgggttaatacctcgggaggatgacggtacctgaagaataagcaccggctaactacgtgccagcagccgcggtaatacgtagggtgcaagcgttaatcggaattactgggcgtaaagcgtgcgcaggcggttatgtaagacagatgtgaaatccccgggcttaacctgggaactgcatttgtgactgcatagcttgagtgcggcagagggggatggaattccgcgtgtagcagtgaaatgcgtagatatgcggaggaacaccgatggcgaaggcaatcccctgggcctgcactgacgctcatgcacgaaagcgtggggagcaaacaggattagataccctggtagtccacgccctaaacgatgtcaactggttgttgggagggtttcttctcagtaacgtagctaacgcgtgaagttgaccgcctggggagtacggccgcaaggttgaaactcaaaggaattgacggggacccgcacaagcggtggatgatgtggtttaattcgacgcaacgcgaaaaaccttacctacccttgacatgccagggatcctgcagagatgtgggagtgctcgaaagagaacctggacacaggtgctgcatggccgtcgtcagctcgtgtcgtgagatgttgggttaagtcccgcaacgagcgcaacccttgtcattagttgctacgtaagggcactctaatgagactgccggtgacaaaccggaggaaggtggggatgacgtcaggtcatcatggcccttatgggtagggctacacacgtcatacaatggccggtacagagggctgccaacccgcgagggggagccaatcccagaaaaccggtcgtagtccggatcgcagtctgcaactcgactgcgtgaagtcggaatcgctagtaatcgcggatcagcttgccgcggtgaatacgttcccgggtcttgtacacaccgcccgtcacaccatgggagcgggttctgccagaagtagttagcctaaccgcaaggagggcgattaccacggcagggttcgtgactggggtgaagtcgtaacaaggta

SEQ ID NO: 8 - DNA, 1391 bp, Methyloversatilis
agagtttgattatggctcagattgaacgctggcggaatgctttacacatgcaagtcgagcggcagcacgggggtaaccctggtggcgagcggcgaacgggtgagtaatacatcggaacatgcccagtcgtgggggataacacttcgaaagaagtgctaataccgcatacgtcctgagggagaaagcgggggatcgcaagacctcgcgcgattggagtggccgatgtcagattagctagttggtggggtaaaggcctaccaaggcaacgatctgtagctggtctgagaggatgatcagccacactgggactgagacacggcccagactcctacgggaggcagcagtggggaattttggacaatgggggcaaccctgatccagccattccgcgtgagtgaagaaggccttcgggttgtaaagctctttcggcaggaacgaaacggtgagctctaacatagcttgctaatgacggtacctgaagaagaagcaccggctaactacgtgccagcagccgcggtaatacgtagggtgcgagcgttaatcggaattactgggcgtaaagcgtgcgcaggcggttgtgtaagacaggtgtgaaatccccgggcttaacctgggaactgcgcttgtgactgcacagctagagtatggcagaggggggtggaattccacgtgtagcagtgaaatgcgtagagatgtggaggaacaccgatggcgaaggcagccccctgggccaatactgacgctcatgcacgaaagcgtggggagcaaacaggattagataccctggtagtccacgccctaaacgatgtcgactaggtgttcggtgaggagactcattgagtaccgcagctaacgcgtgaagtcgaccgcctggggagtacggtcgcaagattaaaactcaaaggaattgacggggacccgcacaagcggtggatgatgtggattaattcgatgcaacgcgaaaaaccttacctacccttgacatgtacggaaccctgctgagaggtgggggtgctcgaaagagagccgtaacacaggtgctgcatggctgtcgtcagctcgtgtcgtgagatgttgggttaagtcccgcaacgagcgcaacccttgtcattagttgctacgcaagagcactctaatgagactgccggtgacaaaccggaggaaggtggggatgacgtcaagtcctcatggcccttatgggtagggcttcacacgtcatacaatggtcggtacagagggttgccaagccgcgaggtggagccaatcccagaaagccgatcgtagtccggatcgcagtctgcaactcgactgcgtgaagtcggaatcgctagtaatcgcggatcagcatgccgcggtgaatacgttcccgggtcttgcacacaccgcccgtc

SEQ ID NO: 9 - DNA, 1412 bp, Rhodopseudomonas
agcgaacgctggcggcaggcttaacacatgcaagtcgaacgggcgtagcaatacgtcagtggcagacgggtgagtaacgcgtgggaacgtaccttttggttcggaacaacacagggaaacttgtgctaataccggataagcccttacggggaaagatttatcgccgaaagatcggcccgcgtctgattagctagttggtgaggtaatggctcaccaaggcgacgatcagtagctggtctgagaggatgatcagccacattgggactgagacacggcccaaactcctacgggaggcagcagtggggaatattggacaatgggggaaaccctgatccagccatgccgcgtgagtgatgaaggccctagggttgtaaagctcttttgtgcgggaagataatgacggtaccgcaagaataagccccggctaacttcgtgccagcagccgcggtaatacgaagggggctagcgttgctcggaatcactgggcgtaaagggtgcgtaggcgggtttctaagtcagaggtgaaagcctggagctcaactccagaactgcctttgatactggaagtcttgagtatggcagaggtgagtggaactgcgagtgtagaggtgaaattcgtagatattcgcaagaacaccagtggcgaaggcggctcactgggccattactgacgctgaggcacgaaagcgtggggagcaaacaggattagataccctggtagtccacgccgtaaacgatgaatgccagccgttagtgggtttactcactagtggcgcagctaacgctttaagcattccgcctggggagtacggtcgcaagattaaaactcaaaggaattgacgggggcccgcacaagcggtggagcatgtggtttaattcgacgcaacgcgcagaaccttaccagcccttgacatgtccaggaccggtcgcagagacgcgaccttctcttcggagcctggagcacaggtgctgcatggctgtcgtcagctcgtgtcgtgagatgttgggttaagtcccgcaacgagcgcaacccccgtccttagttgctaccatttagttgagcactctaaggagactgccggtgataagccgcgaggaaggtggggatgacgtcaagtcctcatggcccttacgggctgggctacacacgtgctacaatggcggtgacaatgggaagctaaggggcgacccttcgcaaatctcaaaaagccgtctcagttcggattgggctctgcaactcgagcccatgaagttggaatcgctagtaatcgtggatcagcatgccacggtgaatacgttcccgggccttgtacacaccgcccgtcacaccatgggagttggctttacctgaagacggtgcgctaaccagcaagtgggggcagccggccacggtagggtcagcgactggggtg